(19) United States
(12) Patent Application Publication
(10) Pub. No.: US 2022/0016321 A1
(43) Pub. Date: Jan. 20, 2022

(54) DERMAL SUBSTITUTES AND ENGINEERED SKIN WITH RETE RIDGES

(71) Applicant: Ohio State Innovation Foundation, Columbus, OH (US)

(72) Inventors: Heather POWELL, Mason, OH (US); Britani BLACKSTONE, Columbus, OH (US); John BAILEY, Clemmons, NC (US)

(21) Appl. No.: 17/413,803

(22) PCT Filed: Dec. 13, 2019
(86) PCT No.: PCT/US2019/066359

§ 371 Date: Jun. 14, 2021

Related U.S. Application Data
(60) Provisional application No. 62/779,276, filed on Dec. 13, 2018.

(51) Int. Cl. A61L 27/60; A61L 27/38; A61L 27/24

(52) U.S. Cl. CPC A61L 27/60; A61L 27/3813; A61L 2300/414; A61L 27/24; A61L 2400/18; A61L 27/3886

(57) ABSTRACT

Disclosed herein are dermal substitutes comprising: fibroblasts positioned in a biologically compatible matrix, the biologically compatible matrix comprising a plurality of protrusions on at least one surface; wherein the plurality of protrusions comprise a length and width sufficient to improve a dermal graft outcome. Also disclosed are methods of treating a skin wound on a subject, the method comprising: contacting a skin wound with a herein disclosed dermal substitute. Also disclosed are methods of preparing a dermal graft for transplantation, the method comprising: culturing fibroblasts positioned in a biologically compatible matrix in a scaffold comprising a plurality of protrusions on at least one surface; wherein the scaffold comprises a plurality of protrusions comprising a length and width sufficient to improve a dermal graft outcome.

### CROSS-REFERENCE TO RELATED APPLICATIONS

This application claims benefit of priority of U.S. Provisional Application No. 62/779,276, filed Dec. 13, 2018, which is hereby incorporated by reference in its entirety.

### FIELD

The disclosure generally relates to compositions and methods for treating skin disease and defects using grafts or transplants.

### BACKGROUND

Prompt closure of wounds in the 30,000 children suffering massive (>70% Total Body Surface Area; TBSA) burn injuries annually in the United States (Sheridan et al., JAMA, 2000; 283:69-73) is critical to prevention of infection and survival. A major limiting factor to treating these wounds is a lack of donor sites for treatment with split-thickness autograft. A number of bioengineering strategies have been employed to overcome the challenges associated with these injuries. Despite significant positive outcomes with auto (Boyce et al., J Trauma. 2006; 60(4):821-9; Supp et al., Clin Dermatol. 2005; 23(4):403-12; Boyce et al., Am J Surg. 2002; 183(4):445-56; Boyce et al., Ann Surg. 2002; 235(2):269-79; Llames et al., Cell Tissue Bank. 2006; 7:47-53; Khadjibayev et al., 2008; 21(3):150-152) and chimeric allo-/auto-engineered skin grafts (Domres et al., Ann Bums Fire Disasters. 2007; 20(3):149-154; Rasmussen et al., Mil Med. 2014; 179(8 Suppl):71-8; Schurr et al., Adv Wound Care (New Rochelle). 2012; 1(2):95-103; Centanni et al., Ann Surg. 2011; 253(4).672-683; Schurr et al., J Trauma. 2009; 66(3):866-874), these technologies are not yet commercially available (Boyce et al., J Trauma. 2006; 60(4):821-9; Schurr et al., Trauma. 2009; 66(3):866-874; Williams et al., Biotechnol J. 2014; 9(3):337-347). Cultured epithelial grafts (autografts or allografts, collectively “CEA”; sheets of keratinocytes cultured in vitro) are commercially available (Epiceirm from Genzyme Corp.) and are indicated for the treatment of massive bum injuries in both children and adults. Though success with this therapy has been reported from a small number of bum centers (Sood et al., J Bum Care Res. 2010; 31(4):559-68; Clugston et al., J Burn Care Rehabil. 1991; 12(6):533-9; Carsin et al., Burns. 2000; 26(4):379-87; McAree et al., J Pediatr Surg. 1993; 28(2):166-168), a large number of others report poor outcomes due to graft fragility, difficulty in surgical application, blistering, poor engraftment rates, infection and significant contracture (FIG. 1) (Gobet et al., Surgery. 1997; 121(6):654-61; Wood et al., Burns. 2006; 32(4):395-401; Atiyeh et al., Burns. 2007; 33(4):405-413; Desai et al., J Burn Care Rehabil. 1991; 12(6):540-5; Barret et al, Ann Surg. 2000; 231(6):869-876; Compton, Skin Research. 1996; 38(1):148-159; Longaker M T., Scarless Wound Healing. New York: Dekker; 2000). Despite significant challenges with this therapy and frequent suboptimal results, CEAs continue to be utilized due, in part, to the absence of other treatment options for massive burn wounds. As poor outcomes with CEA are commonly associated with its extreme fragility, blistering and poor adhesion to the wound bed which can persist years post grafting.

Within the human body, fibroblasts are responsible for the majority of extracellular matrix (ECM) deposition and matrix remodeling. Fibroblasts represent a heterogeneous population of cells with differing shapes, sizes, remodeling capabilities and potential to cause fibrosis (Rinkevich et al., Science. 2015; 17; 348(6232):aaa2151). For example, the depth of fibroblasts within the dermis dictated their function with fibroblasts isolated from the papillary dermis exhibiting increased plating efficiencies, faster growth rates, and less contact inhibition than reticular fibroblasts (Tajima et al., J Invest Dermatol. 1981; 77(5):410-2; Sorrell et al., J Cell Physiol. 2004; 200(1):134-45). In addition, papillary fibroblasts have increased decorin, collagenase and type XVI collagen expression (Schonherr et al., Biochem J. 1993; 290(Pt3):893-99; Ali-Bahar et al., Wound Repair Regen, 2004; 12(2):175-82; Akagi et al., J invest Dermatol. 1999; 113(2):246-50; Baker et al., Biomacromolecules. 2011; 12(4997-1005), Fibroblasts from the papillary dermis also induce different responses from keratinocytes. Keratinocytes co-cultured with papillary fibroblasts produced filaggrin and type VII collagen that was absent from keratinocytes cultured with reticular fibroblasts.

Cellular crosstalk between the epithelium and mesenchyme is known to regulate epidermal homeostasis, morphogenesis and basement membrane formation (Fleischmajer et al., J Cell Sci. 1998; 111 (Pt 14):1929-40; Smola et al., Exp Cell Res. 1998; 239(2):399-410; Maas-Szabowski et al., J Cell Sci. 1999; 11.12(Pt 12):1843-53; Mackenzie I C., The Keratinocyte Handbook. Cambridge: Cambridge University Press; 1994; Fusenig N E., The Keratinocyte Handbook. Cambridge: Cambridge University Press; 1994). Nutrient exchange between the epidermis and dermis at the rete ridges has been proposed to facilitate healthy epidermal function and create an interface where significant chemical communication between the two layers can take place. The double paracrine loop of IL-1α/IL-1β from keratinocytes and keratinocyte growth factor (KGF; also called :FGF7) or GM-CSF from fibroblasts has been shown to be a key component of epidermal-dermal communication and epidermal homeostasis (Maas-Szabowski et al., J Cell Sci. 1999; 112(Pt 12):1843-53; Szabowski et al., Cell, 2000; 103(5):745-55; Maas-Szabowski et al., J Invest Dermatol. 2001; 116(5):816-20). More recently it was proposed that keratinocyte proliferation and differentiation depend on an autocrine loop of periostin within fibroblasts (Taniguchi et al., J Invest Dermatol. 2014; 134(5):1295-304), where periostin produced by fibroblasts cooperates with IL-1a from keratinocytes to synergistically induce the NF-κB pathway and produce IL-6. In organotypic models for skin, the presence of both fibroblasts and keratinocytes was needed for basement membrane formation (Andriani et al., J Invest Dermatol. 2003; 120:923-31), the formation of a continuous densely packed basal cell layer (Erdag et al., Burns. 2004; 30(4):322-8), and the maintenance of the stratified epidermis after transplantation (Thokuchi et al., Cell Tissue Res. 1995; 281(2):223-9). These results suggest that epithelial-mesenchymal interactions are key regulators of epidermal development and maintenance and that providing the appropriate physical environment for interaction may enhance communication and improve epidermal viability and homeostasis within a graft,

In normal human skin (NHS) the dermal-epidermal interface is interdigitated to facilitate a strong connection between the two layers. In humans, areas routinely exposed to significant amounts of mechanical shear (e.g., soles of feet, palms) have rete ridges with greater density and depth (Odland G F., Anat Record. 1950; 108(3).399-413). In cultured epithelial grafts, rete ridge formation after grafting is extremely slow to form with no rete ridge structures observed up to 3 years post-engraftment in 75% of patients (Putland et al., J Burn Care Rehabil. 1995; 16(6):627-640). When rete ridges did form, they were fewer in number and flatter in comparison to split-thickness autografts which was proposed to be a primary cause of the poor mechanical properties and blistering associated with cultured epithelial grafts (Atiyeh et al., Burns. 2007; 33(4):405-413; Putland et al., J Burn Care Rehabil. 1995; 16(6):627-640; Leary et al., J invest Dermatol. 1992; 99(4):422-30). The presence of channels in epidermal analogs has been shown to upregulate basement membrane protein deposition at the corners and bottom of the channels (Downing et al., J Biomed Mater Res A. 2005; 72(1):47-56).

Challenges to engraftment include a shift in expressed surface integrins during the culture process from those associated with adhesion and proliferation to those associated with differentiation (Atiyeh et al., Burns. 2007; 33(4):405-413). In addition, the use of di spase and other enzymatic processes to remove the cells from the tissue culture vessel reduce the graft's ability to rapidly adhere to the wound bed (Atiyeh et al., Burns. 2007; 33(4):405-413). At the time of grafting, no basal lamina, mature hemidesmosomes or anchoring fibrils are present at the attachment face of a graft such as a CEA (Compton, Skin Research. 1996; 38(1):148-159; Compton et al., Lab Invest. 1989; 60(5):600-12). It is not until weeks 3-4 post-grafting where basal lamina. confluency, hemidesmosome maturation and thickening of anchoring fibrils are observed; however, rete ridges are still absent (Compton, Skin Research. 1996; 38(1):148-159; Compton et al., Lab Invest. 1989; 60(5):600-12). Furthermore, CEA sheets remain fragile for up to 1 year post engraftment with ulceration or spontaneous blistering occurring after minimal trauma (Atiyeh et al., Burns. 2007; 33(4):405-413; Longaker M T., Scarless Wound Healing. New York: Dekker; 2000). Due to incomplete basement membrane structures and abnormal anchoring fibrils and rete ridge formation at the epidermal-wound junction following CEA engraftment, the epidermis generated by CEAs are weaker than normal skin with lower resistance to shear forces and a high susceptibility to breakdown (Compton, Skin Research. 1996; 38(1):148-159; Longaker M T., Scarless Wound Healing. New York: Dekker; 2000; Leary et al., J Invest Dermatol. 1992; 99(4):422-30). The loss of the CEA healed surface with blistering is thought to be associated with secondary delayed healing and poor scar outcome (Desai et al., J Burn Care Rehabil. 1991; 12(6):540-5). In addition, contracture of the scar is a problem with the fragile surface making scar management more difficult (Wood et al., Burns. 2006; 32(4)395-401). Thus, improvements to CEA durability, engraftment and resistance to shear force will be critical to improving functional outcomes for these patients.

The compositions and methods disclosed herein address these and other needs.

### SUMMARY

Disclosed herein is a dermal substitute containing engineered rete ridges, which can be applied to a skin wound for improved skin healing or regrowth. The dermal substitute can also serve as a carrier for skin grafts and transplants, including cultured epithelial autografts and cultured epithelial allografts (collectively, “CEA”). The dermal template with rete ridges can not only facilitate surgical application but also enhance epidermal-dermal communication, increase basement membrane protein deposition, enhance interfacial strength, reduce graft contraction, among other benefits.

Dermal substitutes with engineered rete ridges can be fabricated with tunable rete ridge depth, width, frequency and density. The form of the rete ridges can be tightly controlled and used as an acellular scaffold (FIG. 2). a fibroblast seeded dermal substitute (FIG. 4), or the dermal component of a full-thickness skin substitute and/or a cultured epithelial autograft (FIG. 8; FIG. 9).

Autologous porcine CEAs exhibited lower engraftment rates, significant contraction and poor rete ridge formation 30 days post-grafting (FIG. 11; FIG. 12). For the first time, autologous porcine CEAs containing engineered rete ridges were successfully used to treat a full-thickness porcine burn wound model (Philandrianos et al., Burns. 2012; 38(6):820-9; reporting 0% engraftment). The disclosed dermal substitute compositions performed better in engraftment studies compared to simply transferring keratinocytes or keratinocytes on a membrane, as disclosed in van den Bogaerdt et al., Wound Repair Regen. 2004; 12(2):225-34; Bevan et al., Burns. 1997; 23(7-8):525-32).

Engineered rete ridges increased keratinocyte growth factor (KGF) and interleukin 6 (IL-6) production in a full-thickness engineered skin model (FIG. 9). IL-6 and KGF are known to increase keratinocyte proliferation in culture. As epithelial-mesenchymal communication is needed for epidermal homeostasis and morphogenesis, providing an enhanced surface for interaction can improve epidermal viability. CEA often exhibits a shift in the natural epidermal homeostasis towards a more differentiated phenotype. A restoration to a more proliferative phenotype can improve adhesivity to the dermis and prevent loss and blistering.

In one aspect, disclosed herein is a dermal substitute comprising a substantially planar sheet comprising fibroblasts dispersed within a biocompatible polymer matrix. The substantially planar sheet can comprise a plurality of protrusions extending from at least one surface of the substantially planar sheet. The plurality of protrusions can be sized (e.g., can have a height, length and/or width, spacing, and shape) to improve a dermal graft outcome. In some embodiments, the substantially planar sheet can comprise an inner surface and an outer surface, and the plurality of protrusions can extend from the inner surface of the substantially planar sheet. In some embodiments, the biologically compatible matrix comprises collagen. The collagen can have a sufficient porosity to permit the diffusion of nutrients through the matrix to fibroblasts dispersed within the matrix.

In some embodiments, each of the plurality of protrusions has a length of at least 50 μm, such as a length of from 50 μm to 750 μm. In some embodiments, each of the plurality of protrusions has a width of at least 50 μm, such as a width of from 50 μm to 750 μm. In some embodiments, each of the plurality of protrusions has a height of at least 10 μm, such as a height of from 10 μm to 750 μm. In some embodiments, the plurality of protrusions spaced apart by an average spacing along an axis of least 50 μm, such as a width of from 50 μm to 750 μm.

In some embodiments, the dermal graft outcome can comprise: increasing a rate of epidermal barrier formation, increasing an amount of rete ridge formation, increasing an amount of epidermal-dermal tissue chemical communication, increasing an amount of basement membrane protein deposition, increasing an amount of interfacial strength, decreasing an amount of transepidermal water loss (TEWL), decreasing an amount of graft contraction, or a combination thereof.

In some embodiments, the dermal substitute can further comprise a population of epithelial cells disposed on the outer surface of the substantially planar sheet. In some embodiments, the population of epithelial cells can comprise a skin graft, such as an autologous skin graft, an isogenic skin graft, an allogenic skin graft, or a xenogenic skin graft. In some embodiments, the population of epithelial cells can be cultured on the outer surface of the substantially planar sheet. In some embodiments, the cells can be applied to the outer surface as a spray. For example, the spray can be a dispersion of epithelial cells such as those prepared using a system to apply autologous stem cells, such as the system commercially available from Avila. Medical under the tradename RECELL®.

Also disclosed are methods of treating a skin wound on a subject that comprise contacting the skin wound with a dermal substitute described herein. As described above, in some embodiments, the dermal substitute can comprise a substantially planar sheet comprising an inner surface and an outer surface, and a plurality of protrusions extending from the inner surface of the substantially planar sheet. In these embodiments, methods of treating a skin wound can comprise positioning the inner surface of the substantially planar sheet in contact with the skin wound.

In some embodiments, the skin wound comprises a bum. In some embodiments, the dermal substitute can further comprise a population of epithelial cells disposed on the outer surface of the substantially planar sheet. In some embodiments, the population of epithelial cells can comprise a skin graft, such as an autologous skin graft, an isogenic skin graft, an allogenic skin graft, or a xenogenic skin graft. In some embodiments, the population of epithelial cells can be cultured on the outer surface of the substantially planar sheet prior to positioning the dermal substitute in contact with the skin wound. In some embodiments, the method can increase an amount of a skin growth or repair factor comprising any one or more of keratinocyte growth factor (KGF), IL-6, IL-1a, IL-1b, periostin, Ki67, involucrin, loricrin, p63, β1 integrin, keratin 5, keratin 14, delta 1 or connexin 43. In some embodiments, the cells can be applied to the outer surface as a spray. For example, the spray can be a dispersion of epithelial cells such as those prepared using a system to apply autologous stem cells, such as the system commercially available from Avita Medical under the tradename RECELL®.

Also provided are methods of preparing a dermal graft for transplantation. The methods can comprise culturing fibroblasts dispersed within a biocompatible polymer matrix. The biocompatible polymer matrix can be formed as a substantially planar sheet having an inner surface and an outer surface. The inner surface of the substantially planar sheet can comprise a plurality of protrusions extending from the inner surface of the substantially planar sheet.

In some embodiments, methods can further comprise forming the biocompatible polymer matrix into the form of a substantially planar sheet having an inner surface and an outer surface, wherein the substantially planar sheet comprises a plurality of protrusions extending from the inner surface of the substantially planar sheet. In some embodiments, the methods can further comprise crosslinking the collagen matrix. In some embodiments, the methods can further comprise exposing the collagen matrix to a laser delivering at least 5 mJ.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIG. 1(A-F) are photographs of CEAs (Epicel™, Genzyme Corp.) placed onto pediatric burn patients. (A-C) CEA is extremely fragile and must be grafted using a petroleum gauze backer. Outcomes with CEA vary greatly from significant graft loss (D) to high engraftment and maintenance of skin pliability (E-F).

FIG. 2(A-F) are images showing scaffold architecture. A,D) Photographs of collagen scaffolds with rete ridges engineered into their surfaces. Hydrated, acellular scaffolds are durable and can be easily handled. Scanning electron microscope (SEM) images of flat (B), 250×250×200 μm (1×w×d, C), 500×500×200 μm (E) and 800×800×200 μm (F) rete ridges.

FIG. 3 is a schematic of photolithography technique used to engineer rete ridges.

FIG. 4 are H&E stained histological sections of dermal substitutes with flat (A) or fete ridge surfaces (B-D).

FIG. 5 includes SEM images of an engineered rete ridge showing differences in collagen organization between the sidewall of the well and the bottom of the well/surrounding collagen.

FIG. 6(A-C) shows A,B) immunocytochemistry of fete ridge containing dermal substitutes showing macrophage clustering around the rete ridges. C) Analysis of inflammatory cytokine production via ELISA shows slight increases in MCP-1 and TGF-β with the presence of rete ridges but little difference in IL-6 or IL-8 production.

FIG. 7(A-I) is a set of images showing procedures for porcine burn wound (A-B), eschar removal (C), split-thickness autograft (D) or dermal substitute (E)/CEA application (F) followed by bolster securing (G), fiberglass protective shell (H) and final dressings

FIG. 8(A-B) is a set of images showing A) the current state of the art in rete ridge formation where keratinocytes and fibroblasts are seeded on the opposite side of a thin film covered collagen sponge (Clement et al., Acta Biomater. 2013; 9(12):9474-84). B) A new dermal substitute-CEA/epidermal model has direct contact and interaction between the epidermis and dermis.

FIG. 9(A-D) shows H&E stained sections of engineered skin constructed with rete ridges (A&B). Production of (C) and KGF (D) was increased in engineered skin containing rete ridges compared to flat controls.

FIG. 10 shows A) H&E section of a control, porcine burn wound after 70 days. B) Image of burn wound in same pig after 70 days but after daily, topical tocotrienol to the wound to induce rete ridge formation. Delamination of the epidermis and dermis were observed in the control wound (C, arrows). Rete ridge formation prevented delamination events during testing of the tocotrienol treated wounds (D).

FIG. 11 shows left panel: photographs of CEA and CEA+collagen on porcine burn wounds 7 days post injury. Stable epithelium can be observed by the continuous matte appearance of the skin (white arrows) with graft loss seen where the wound bed is visible (dark arrows). Right panel: Percent graft take at 7 days for CEA was highly variable, ranging from 0-60% with an average of 38%. In contrast, engraftment rates were higher and more uniform when CEA was combined with a rough collagen dermal template.

FIG. 12 shows H&E stained sections of porcine CEA (A) and CEA+Collagen grafts (B) 28 days after grafting. CEA+collagen grafts had (C) lower rates of transepidermal water loss (TEWL) and (D) less contraction at days 14 and 28/30.

FIG. 13 shows the morphology of the dermal component assessed following laser treatment and compared to untreated, flat, samples. SEM displayed a uniform layer of tightly packed fibroblasts on flat samples, while ridged samples presented a similar cell layer broken up by laser ablated regions surrounded by areas of damaged cells and collagen fibers.

FIG. 14 shows the dermal template with and without ridges. Templates were immunostained with phalloidin to visualize the cytoskeleton of the dermal fibroblasts (green) with DAN utilized to identify the nuclei of the dermal fibroblasts (blue). Fibroblasts are randomly arranged in the flat template whereas they become more concentrated around the rete ridges.

FIG. 15 shows immunostained histological sections of engineered skin fabricated with flat and ridged templates. Staining for cell nuclei (blue), laminin-5 (a basement membrane protein, green) and cytokeratin (red, identifies keratinocytes) show the interdigitate structure of engineered skin made with the ridged template along with a greater epidermal area and significant basement membrane protein deposition.

FIG. 16 shows a MTT cell viability assay for engineered skin made with a flat dermal template and a ridges dermal template. The assay shows that the process utilized to fabricate the rete ridges does not significantly reduce cell viability.

FIG. 17 shows in vitro measurements of surface electrical capacitance (SEC) of engineered skin fabricated with flat and ridged dermal templates. SEC values are inversely related to the establishment of epidermal barrier function. Ridges templates result in lower SEC values at culture day 11 suggesting an increase in epidermal differentiation and barrier formation versus flat templates.

FIG. 18 shows athymic mice grafted with human engineered skin fabricated with flat or ridges dermal templates. Engineered skin fabricated with ridges dermal templates results in high levels of engraftment and equivalent levels of graft contraction.

FIG. 19 shows transepidermal water loss (TEWL) measurements from engineered skin grafted to athymic mice. At two week post-grafting flat and ridged groups have similar values of water loss; however by week 4, the engineered skin made with a ridged template has significantly reduced TEWL compared to flat controls.

FIG. 20 shows immunostained histological sections of human engineered skin made with flat and ridged dermal templates. Proliferating cells were stained with Ki67 (red), basement membrane with laminin-5 (green), protein within the cornified envelop of the epidermis with loricrin (gray) and cell nuclei with DAPI (blue). Rete ridges were stable and persists until the end of the study with continuous basement membrane observed. Qualitatively, the epidermis appears to have a significant increase in proliferative cells in the engineered skin made with a ridged template.

FIG. 21 shows quantitative analyses of basement membrane area, epidermal area per field of view and number of Ki67+keratinocytes per field of view in flat and ridged skin as a function of time. One week prior to grafting (week −1), rete ridged engineered skin has significantly greater basement membrane and actively proliferating keratinocytes.

Epidermal area was significantly enhanced by the presence of rete ridges following grafting until the end of the study.

FIG. 22 shows biomechanical properties of flat and ridged engineered skin. Average skin strength and toughness was greater in ridged skin; however, only increases in linear stiffness with the presence of rete ridged were statistically significant.

FIG. 23 shows the evolution of rete ridge formation following laser ablation. Skin was immunostained with DAPI (all nuclei, blue), cytokeratin (all keratinocytes, red) and involucrin (all layers of the epidermis but the stratum basalae, green).

FIG. 24 shows additional visualization of ridge formation showing basement membrane protein, collagen IV (green), involucrin (red) and cell nuclei (blue).

FIGS. 25A and 2B show graft contraction in flat and ridged skin over 4 weeks. As shown in FIG. 25B, torsional ballistometry was performed on the grafts to non-invasively measure graft biomechanics. No significant differences were observed between flat and ridged grafts.

FIG. 26 shows H&E stained sections of epithelial sheets grown in the laboratory versus manufactured commercially.

FIG. 27 shows photographs of mice with full-thickness cutaneous injuries treated with CEA in conjunction with a flat dermal template (0 mJ), or ridged templates (5 mJ, 25% density or 50 mJ p4 setting).

FIG. 28 shows H&E stained sections of grafted CEA+dermal substitutes at weeks 2 and 4 post-grafting.

FIG. 29 shows image analysis of epidermal area per field of view for CEAs grafted on flat and ridged dermal templates. The ridges template at 5 mJ and 25% density significantly improved epidermal area versus 50 mJ p4 and flat controls.

FIG. 30 shows quantification of the number of delamination events that occurred during the tensile testing of skin healed with CEA+dermal templates. On average there were less delamination events when the 5 mJ 25% ridged dermal template was used.

FIG. 31 shows that this process can be utilized to form an interdigitated interface in other tissues including engineered gingiva.

FIG. 32 shows fibroblast seeded collagen scaffolds laser ablated with increasing laser powers (A->C, D->F) and with two different laser heads. Scale bar=100 μm

FIG. 33 shows the maximum load and linear stiffness of engineered skin with a flat or ridges epidermal-dermal interface. Engineered skin was tensile tested to failure at 2 and 4 weeks post-grafting to an athymic mouse with stents applied to the peri-graft area to more closely mimic the natural tension on human skin.

FIG. 34 is a schematic illustration of the formation of different dermal substitutes.

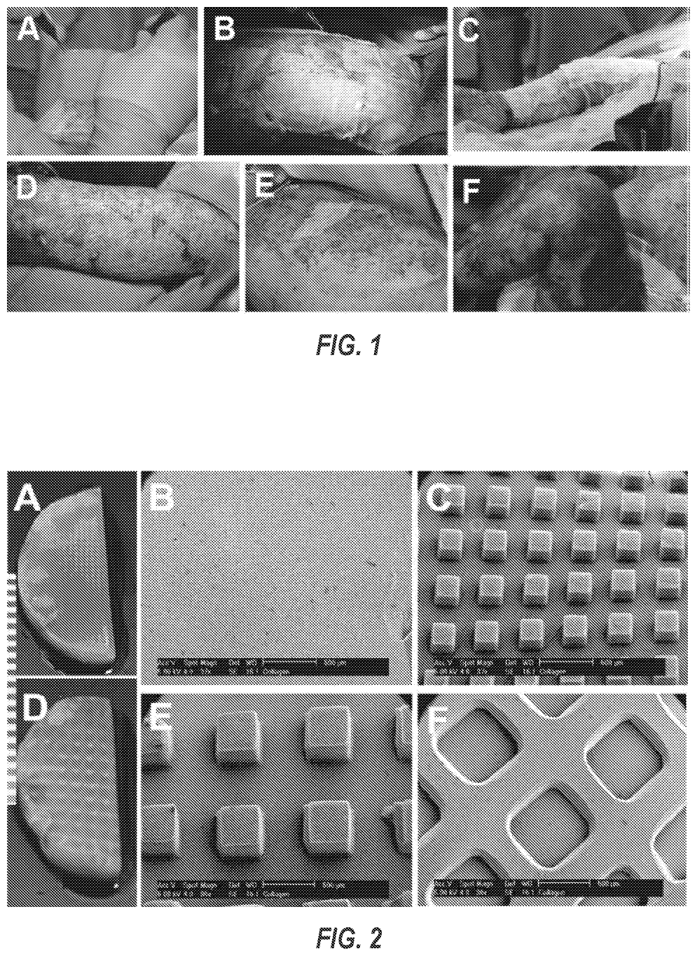

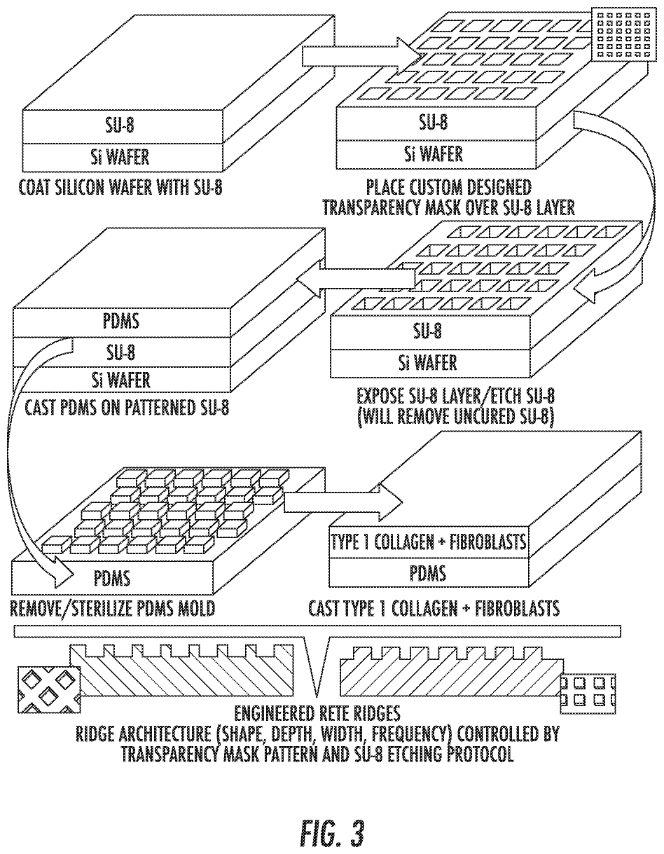

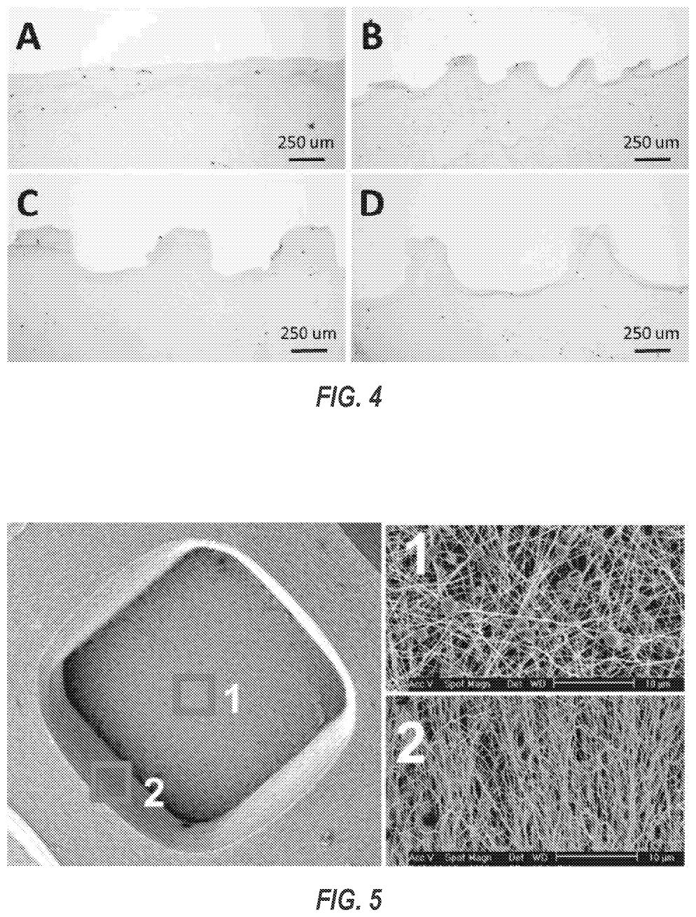

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular dermal substitute is disclosed and discussed and a number of modifications that can be made to the dermal substitute are discussed, specifically contemplated is each and every combination and permutation of the dermal substitute and the modifications that are possible unless specifically indicated to the contrary. Thus, if a class of dermal substitutes A, B, and C are disclosed as well as a class of dermal substitutes D, E, and F and an example of a combination dermal substitute, or, for example, a combination dermal substitute comprising A-D is disclosed, then even if each is not individually recited each is individually and collectively contemplated meaning combinations, A-E,k-F, B-D, B-E, B-F, C-D, C-E, and C-F are considered disclosed. Likewise, any subset or combination of these is also disclosed. Thus, for example, the sub-group of A-E, B-F, and. C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

### Compositions

It is understood that the dermal substitutes of the present disclosure can be used in combination with the various compositions, methods, products, and applications disclosed herein,

Disclosed herein are dermal substitute compositions comprising: fibroblasts positioned in a biologically compatible matrix (e.g., a collagen matrix), the biologically compatible matrix comprising a plurality of protrusions on at least one surface; wherein the plurality of protrusions are sized (e.g., have a length, width, and/or height) sufficient to improve a dermal graft outcome.

As used herein, the term “dermal substitute” refers to any artificially formed, biologically compatible matrix comprising fibroblast cells usable for grafting or transplanting onto a subject's skin for purposes of wound healing or skin growth/repair.

A sufficient amount of fibroblasts are included in the biologically compatible matrix to promote skin growth or repair after application to a subject, or to produce a culturable graft which can be cultured or stored for future use as or with a skin graft or transplant. In some embodiments, at least 1,000, at least 10,000, at least 50,000, at least 100,000, at least 200,000, at least 250,000, at least 300,000, at least 400,000, at least 500,000, or more fibroblast cells/cm² are included in the matrix. In some embodiments, a low amount of fibroblast cells (e.g., about 100) can be included in the matrix and cultured and expanded prior to use.

The matrix can further include additional cell types in addition to fibroblasts. For instance, other mesenchymal stern cells, keratinocytes, macrophages, adipocytes, melanocytes, Langerhans cells, and Merkel cells, can be included in the matrix. Further, the matrix can include additional biological components such as growth factors and cell signaling molecules (e.g., keratinocyte growth factor, platelet-derived growth factor (PDGF), transforming growth factor (TGF), platelet-derived angiogenesis factor (PDAF), vascular endothelial growth factor (VEGF), epidermal growth factor (EGF), growth factor IGF, interleukins), and extracellular matrix components (e.g., laminin, fibrinogen, fibronectin, elastin, glycosaminoglycans, proteoglycans, glycoproteins, vinculin, spectrin, actomyosin, actin).

The biologically compatible matrix is generally porous to allow permeation of cells, fluids, biological components, nutrients, and other materials. The fibroblasts are positioned in the biologically compatible matrix (fibroblasts are within the matrix). Fibroblasts may also be positioned on the surface of the matrix, and can be dispersed within the matrix with relative uniformity or can be heterogeneously dispersed (for instance, positioned within desirable pockets or locales within the matrix). In some embodiments, the matrix has a permeability of at least 1 μm, at least 5 μm, at least 10 μm, at least 20 μm, at least 50 μm, at least 100 μm, at least 250 μm, at least 500 μm, at least 750 μm, at least 1,000 μm, or more. The permeability of the matrix can generally allow for cell growth and expansion, fluid and nutrient flow, but should not be so great as to substantially comprise the required strength, elasticity, and tissue-anchoring required in a dermal substitute. The permeable matrix can be devoid of significant diffusion barriers to facilitate flow of soluble factors such as nutrients. In some embodiments, the biologically compatible matrix is devoid of significant diffusion barriers on a surface which can contact epithelial cells, for instance in an epidermis. Such embodiments facilitate flow of soluble mediators and nutrients between the dermis and epidermis.

The biologically compatible matrix can comprise a number of materials. In some embodiments, the biologically compatible matrix comprises collagen. In some embodiments, the collagen is chemically, enzymatically, or energetically (e.g., via ultraviolet light or heat) crosslinked. The biologically compatible matrix can also comprise a biologically compatible polymer or hydrogel, for example a tissue engineered hydrogel. Biologically compatible polymers and/or hydrogels include, but are not limited to, polyethylene glycol (PEG), poly (2-hydroxyethyl methacrylate (PHEMA), polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as polylactic acid), poly (glycolic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly (alkylene alkylates); hydrophobic polyethers; polyurethanes; polyetheresters; polyacetals; polycyanoactylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers and combinations thereof Biocompatible polymers can also include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of actylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poiy(ethylmethacrylate), poly(butyltnethacrylate), poly(isobutylmethacrylate), poly(hexlmethacrylate), poly(isodecylmethacrylate), poly(lauryl tnethactylate), poly (phenyl tnethacrylate), poly(methyl acrylate), poly(isopropyl acrylate), poly(isobutyl acrylate), poly(octadecyl acrylate), polyethylene, polypropylene, poly(ethylene oxide), poly(ethylene terephthalate), poly(vinyl alcohols), poly(vinyl acetate, poly vinyl chloride polystyrene and polyvinylpryrrolidone, derivatives thereof linear and branched copolymers and block copolymers thereof and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and combinations thereof. In some embodiments, the biologically compatible matrix can further comprise hyaluronic acid or fibrin.

A plurality of protrusions are positioned on at least one surface, or can be positioned on more than one surface. In some embodiments, the protrusions are positioned on an inner surface which can contact a skin wound. The protrusions can extend from the surface of the dermal substitute, thereby mimicking rete ridges.

The plurality of protrusions can vary in physical characteristics such as length, width, and spacing. In some embodiments, the protrusions are substantially uniform. In some embodiments, the protrusions are heterogenous in at least one characteristic selected from length, width, and spacing. In some embodiments, the protrusions have an average length of at least 10 μm, at least 25 μm, at least 50 μm, at least 100 μm, at least 200 μm, at least 250 μm, at least 500 μm, at least 750 μm, at least 800 μm, at least 1,000 μm, or more. In some embodiments, the protrusions have an average width of at least at least 10 μm, at least 25 μm, 50 μm, at least 100 μm, at least 200 μm, at least 250 μm, at least 500 μm, at least 750 μm, at least 800 μm, at least 1,000 μm, or more. In some embodiments, the protrusions have an average spacing between protrusions of at least at least 10 μm, at least 25 μm, at least 50 μm, at least 100 μm, at least 200 μm, at least 250 μm, at least 500 μm, at least 750 μm, at least 800 μm, at least 1,000 μm, or more. As to average length, width, and spacing, the protrusions can contain a range between any disclosed value (e.g., from 10 μm to 1,000 μm, or from 50 μm to 500 μm).

The plurality of protrusions can improve any array of dermal graft outcomes. For instance, the dermal graft outcomes can include, but are not limited to, increased epidermal barrier formation, increased rete ridge formation, increased epidermal-dermal tissue chemical communication, increased basement membrane protein deposition, increased interfacial strength, decreased transepidertnal water loss (TEWL), decreased grafi contraction, decreased graft rejection, increased rate of graft healing, and other such benefits. In some embodiments, the improved dermal graft outcome can be compared to a control, for instance a cultured epithelial cell allograft or a dermal substitute lacking the plurality of protrusions.

In some embodiments, the plurality of protrusions improves a dermal graft outcome, as compared to a control, by at least 10%, at least 20%, at least 25%, at least 50%, at least 75%, at least 100%, at least 150%, at least 200%, at least 250%, at least 500%, or at least 1,000%.

In some embodiments, the dermal substitute can be combined with an additional biological skin graft or transplant, for instance a cultured epithelial cell graft. As an example, a cultured epithelial cell graft can be positioned on an outer surface of the dermal substitute, wherein the inner surface of the dermal substitute contains a plurality of protrusions.

The dermal substitute can further be combined with a variety of wound dressings, medicaments, and other pharmaceutical compounds, particularly when applied to a subject's skin wound.

### Methods of Use

A method of treating a skin wound on a subject, the method comprising: contacting a skin wound with a dermal substitute composition comprising fibroblasts positioned in a collagen matrix, the collagen matrix comprising a plurality of protrusions on at least one surface; wherein the plurality of protrusions comprise a length and width sufficient to improve a dermal graft outcome. The methods can contain any herein disclosed dermal substitute.

The subject can be any mammalian subject, for example a human, cow, horse, mouse, rabbit, dog, monkey, etc. In some embodiments, the subject is a primate, particularly a human. The subject can be a male or female of any age, race, creed, ethnicity, socio-economic status, or other general classifiers.

The fibroblasts can from the same subject (e.g., autograft cells), from a different subject of the same or related species (e.g., allograft cells), or can be from a laboratory or commercial cell line.

The skin wound can include any skin wound which can benefit from a skin cell replacement therapy such as a skin graft or skin transplant. Examples of suitable skin wounds include burn, surgical scar, cut or scrape, areas of excised skin such as diseased skin, chemical bum, infected skin or tissue, and others.

The dermal substitute can be contacted directly on the skin wound with or without additional components such as medicaments, dressings, and other pharmaceutical or medical treatment applications. In some embodiments, epithelial cells can be contacted on an outer surface of the dermal substitute. In such embodiments, the epithelial cells can be contacted to the dermal substitute prior to positioning the dermal substitute on a subject (for instance, in culture or a laboratory or manufacturing setting). Additionally or alternatively, the epithelial cells can be contacted to the dermal substitute after positioning the dermal substitute on a subject, for instance as a second step in a skin graft or transplant procedure. An advantage of positioning epithelial cells on an outer surface of the dermal substitute, wherein the dermal substitute contains protrusions extending from an inner surface, is that the protrusions can mimic the anatomy and physiology of rete ridges, which can serve to improve the overall outcome of an epithelial cell graft or transplant. Useful embodiments including epithelial cells include skin grafts (autografts or allografts, such as a cultured, epithelial cell grafts).

In some embodiments, the cells can be applied to the outer surface as a spray. For example, the spray can be a dispersion of epithelial cells such as those prepared using a system to apply autologous stem cells, such as the system commercially available from Avita Medical under the tradename RECLL®

The methods are advantageous because the methods can improve an array of dermal graft outcomes, including, but not limited to, increased epidermal barrier formation, increased rete ridge formation, increased epidermal-dermal tissue chemical communication, increased basement membrane protein deposition, increased interfacial strength, decreased transepidermal water loss (TEWL), decreased graft contraction, decreased graft rejection, increased rate of graft healing, and other such benefits. In some embodiments, the improved dermal graft outcome can be compared to a control, for instance a cultured epithelial cell graft or a dermal substitute lacking the plurality of protrusions. In some embodiments, the method can increase an amount of a skin growth or repair factor comprising any one or more of keratinocyte growth factor (KGF), IL-6, IL-1a, IL-1b, periostin, Ki67, involucrin, loricrin, p63, β1 integrin, keratin 5, keratin 14, delta 1, or connexin 43.

### Methods of Making

Also disclosed herein are methods of preparing a dermal graft for transplantation, the methods comprising: culturing fibroblasts positioned in a biologically compatible matrix in a scaffold comprising a plurality of protrusions on at least one surface; wherein the scaffold comprises a plurality of protrusions comprising a length and width sufficient to improve a dermal graft outcome.

The biologically compatible matrix can include any herein disclosed matrix. In some embodiments, the biologically compatible matrix comprises collagen. The biologically compatible matrix can be crosslinked, which can serve to increase matrix strength, elasticity, or durability. In some embodiments, the methods can comprise stimulating matrix maturation or expansion. In some embodiments, the methods can comprise stimulating fibrillogenesis, for example, stimulating collagen fibrillogenesis in a collagen matrix.

The methods can include positioning the biologically compatible matrix in a scaffold, which can serve as a mold to shape the biologically compatible matrix. Hence, the scaffold can contain the same or similar physical dimensions as the biologically compatible matrix. The plurality of protrusions can vary in physical characteristics such as length, width, and spacing. In some embodiments, the protrusions are substantially uniform. In some embodiments, the scaffold contains protrusions that are heterogenous in at least one characteristic selected from length, width, and spacing. In some embodiments, the scaffold contains protrusions having an average length of at least 10 μm, at least 25 μm, at least 50 μm, at least 100 μm, at least 200 μm, at least 250 μm, at least 50 μm, at least 750 μm, at least 800 μm, at least 1,000 μm, or more. In some embodiments, the scaffold contains protrusions having an average width of at least at least 10 μm, at least 25 μm, 50 μm, at least 100 μm, at least 200 μm, at least 250 μm, at least 500 μm, at least 750 μm, at least 800 μm, at least 1,000 μm, or more. In some embodiments, the scaffold contains protrusions having an average spacing between protrusions of at least at least 10 μm, at least 25 μm, at least 50 μm, at least 100 μm, at least 200 μm, at least 250 μm, at least 500 μm, at least 750 μm, at least 800 μm, at least 1,000 μm, or more. As to average length, width, and spacing, the protrusions of the scaffold can contain a range between any disclosed value (e.g., from 10 μm to 1,000 μm, or from 50 μm to 500 μm).

The scaffold can be formed from essentially any material capable of forming and containing the biologically compatible matrix, but should not impart deleterious effects on cell growth. For instance, the scaffold can be formed from metal, plastic, polymer, or other suitable materials. In some embodiments, the scaffold comprises a non-permeable polymer, which can serve to house or contain the biologically compatible matrix and added fluids or components. In some embodiments, the scaffold comprises a biologically compatible polymer. In some embodiments, the scaffold comprises polydimethysiloxane (PDMS). In sonic embodiments, the scaffold is a 3D-printed material.

The methods can also include imparting surface roughness to a surface of the dermal substitute. Surface roughness can be imparted by an array of methods, including etching using a laser or fine-point tool (e.g., needle), chemical degradation, enzymatic degradation (e.g., matrix metalloproteinase treatment), or exposure to heat (for example, collagen has a denaturation temperature of about 32-40° C.). In some embodiments, the methods comprise exposing an inner surface of the dermal substitute to a laser delivering at least 1 mJ, at least 2 mJ, at least 5 mJ, at least 10 mJ, or more.

The fibroblasts positioned in the biologically compatible matrix can be cultured in the scaffold for a time sufficient to prepare a dermal substitute which can be applied to a skin wound. For instance, the fibroblasts can be cultured for at least I day, at least 2 days, at least 3 days, at least 4 days, at least 5 days, at least 6 days, at least 7 days, at least 8 days, at least 9 days, at least 10 days, at least 12 days, at least 15 days, at least 20 days, at least 25 days, or more.

In some embodiments, the methods can further include storing the scaffold and matrix in long-term storage, for example cold or cryo storage before use. In some embodiments, the methods can further include contacting the scaffold and matrix with epithelial cells, for instance a cultured epithelial autograft or allograft (CAE).

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Construction of Dermal Substitute

Dermal templates with engineered rete ridges were constructed using photolithography to generate multiple molds with precise microfeatures. The protrusions vary in width, depth, and can contain other varying features. Polydimethysiloxane (PDMS) was cast into this mold to create a stamp. Collagen gels containing fibroblasts were then cast into the PDMS stamp to engineer a dermal template with defined rete ridges. The dermal template was separated from the stamp one day after inoculation and cultured for up to 14 days in custom skin medium (Blackstone et al., Tissue Eng Part A. 2014; 20(19-20):2746-2755) with biopsies collected at days 1, 4, 7, 10 and 14 for analysis of ECM deposition, remodeling, fibroblast phenotype, and dermal biomechanics.

Fibroblast-containing collagen gels containing rete ridges were formed as dermal substitutes, which could be maintained in culture for up to 14 days (FIG. 5).

Methods. Dermal substitutes will be cast into PDMS stamps with varying feature architectures, for example stamps which will result in protrusions having widths of 50, 250, 500, 800 μm; depths of 50, 250, 500, 800 μm; and spacing of 50, 100, 250 μm. Collagen type I gels (GenPhys Inc.) will be fabricated according to manufacturer's protocols with a modulus of 750 kPa. Human primary dermal fibroblasts, isolated following protocols previously described (Boyce et al., Methods Mol Med. 1999; 18:365-89), will be added to the gels prior to casting at a density of about 250,000 cells/ml. Collagen fibrillogenesis requires about 20 minutes, after which fresh Dulbecco's Modified Eagles Medium (DMEM; Sigma) supplemented with 4% fetal bovine serum (FBS; Invitrogen, Portland, Oreg.), 10 μg/mL epidermal growth factor (EGF; Peprotech, Rocky Hill, N.J.), 5 mg/mL insulin (Sigma), 0.5 mg/mL hydrocortisone (FEC; Sigma), 100 mM ascorbic-acid-2-phosphate (Sigma) and 1% penicillin-streptomycin (PSF; Invitrogen). Biopsies will be collected on days 1, 4, 7, 10 and 14 for histology (H&E, Masson's Trichrorne) and immunostaining (picrosirius red staining collagen type I (col I), matrix metalloproteinase I (MMP1), ki67, α-smooth muscle actin (α-SMA), and F-actin). Collagen fibril size and shape will be examined via transmission electron microscopy and image analysis (Starborg et al., Nat Protoc. 2013; 8(7):1433-48), Protein and gene expression will be analyzed via Western blotting and quantitative PCR (qPCR), respectively: Col 1, Col III, MMP, MMP 9, MMP 13, and α-SMA. At days 7 and 14, larger biopsies will be collected for mechanical analysis. 40×5 mm strips will be cut and punched into dogbone-shaped specimens for tensile testing specimens and tested at 2 mm/sec until failure. Ultimate tensile strength, stiffness and pliability will be calculated following methodology previously described (Blackstone et al., Tissue Eng Part A. 2014, 20(19-20):2746-2755; Blackstone et al., Adv Wound Care (New Rochelle). 2012; 1(2):69-74).

### Example 2. Immunological Responses to Dermal Substitute

To test mechanical strain on the skin and to test in a burn+autograft model, immune responses will also be evaluated in a porcine bum, excision and CEA model. Pigs are reported to have strong immune responses to transplanted allogeneic cells (Lamme et al., Wound Repair Regen. 2002; 10(3):152-60).

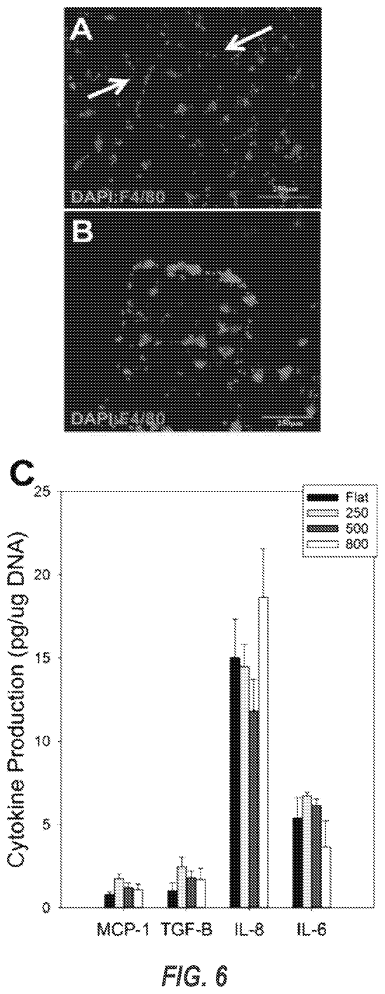

In vitro studies using a human monocyte cell line (THP-1) activated with PMA showed macrophage clustering at ridge edges (FIG. 6A; FIG. 6B) and slight increases in inflammatory cytokines when exposed to dermal substitutes with rete ridges (FIG. 6C).

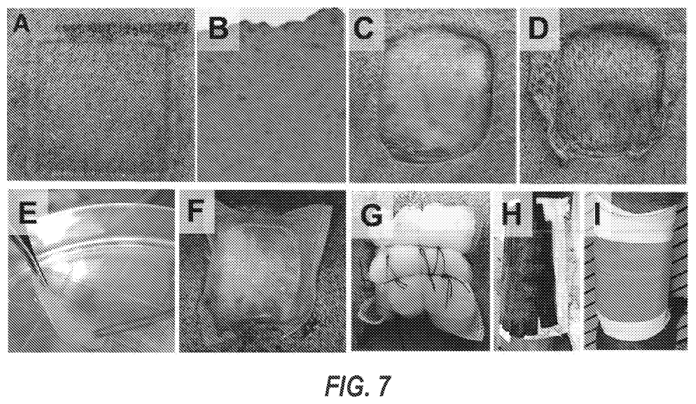

Methods:: Mice will be shaved and their skin disinfected. For each animal, one 10 mm punch will be collected and their fibroblasts isolated and pooled to create a mixed population of allogeneic cells. Following fibroblast expansion and dermal substitute fabrication, one 5 mm incision will be made on the dorsum on both sides of the spine and a 1 cm diameter disk of flat or rete ridge containing collagen matrices or/dermal substitutes will be implanted. Each mouse will receive 2 disks from same group, depth-width-density combinations of width: 50, 250 or 800 μm, depth: 250 μm, spacing: 100 μm. Flat matrices will serve as controls. Incisions will be sutured closed, wounds covered with Tegaderm and dressed with Cohan. Dressings will be removed after 4 days on all animals with groups euthanized at days 4, 7 and 14. Tissue biopsies will be collected for histological analysis and pathological scoring following previously described protocols (Kolb et al., Otolaryngol Head Neck Surg. 2012; 147(3):456-61). In addition, blood samples will be collected at days 1, 4, 7, 10 and 14 and evaluated for white blood cell count, antibodies against the collagen type I gel, and complement-dependent microlymphocytotoxicity against HLA class I antigens following protocol previously described (Falanga et al., Arch Dermatol. 1998; 134(3):293-300). Small (2×2 in) split thickness skin grafts will be collected from each pig and the fibroblasts, isolated, expanded (max p2) and pooled. Full-thickness thermal injuries will be created by placing a 1×1 inch stylus, heated to 200° C., onto the dorsum of Red Duroc pigs for 40 seconds (8 wounds per pig). Burn eschar will be excised, 1×1 inch flat or rete ridge containing allogeneic dermal substitutes (low, medium and high immune responders identified from the murine experiment; sham with no dermal substitute as a control) will be placed into the debrided wound bed followed by a split-thickness autograft meshed and expanded 1:3. Only allogenic dermal substitutes will be examined in this model and each pig will receive only one treatment to prevent cross-contamination between wounds. Grafts will be secured with a bolster dressing and then covered by VetWrap and a fiberglass casting shell to prevent mechanical damage (e.g., FIG. 7). At days 1, 4, 7, 14 and 30, tissue biopsies will be collected from each animal (2 per animal, grafts biopsied only once) and processed for histology and pathology scoring.

### Example 3. Role of Rete Ridge Architecture in Epidermal-Dermal communication and Epidermal Morphogenesis

Rete ridges will be engineered into a dermal substitute. Cultured epithelial autografts (CEAs) will be manufactured following Genzyme Corp. protocols. Following culture, the epithelial layer will be released onto a membrane using Dispase. The CEA will be placed onto the rete ridge dermal substitutes (flat dermal substitutes as a control) and incubated for up to 14 days. Culture medium and biopsies will be collected at different time points and examined for chemical mediators of communication and epidermal viability and differentiation.

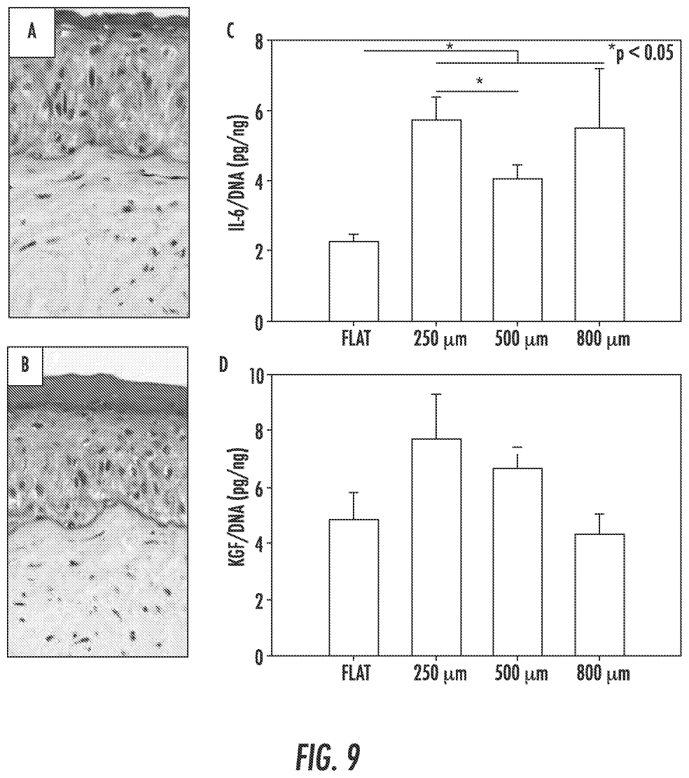

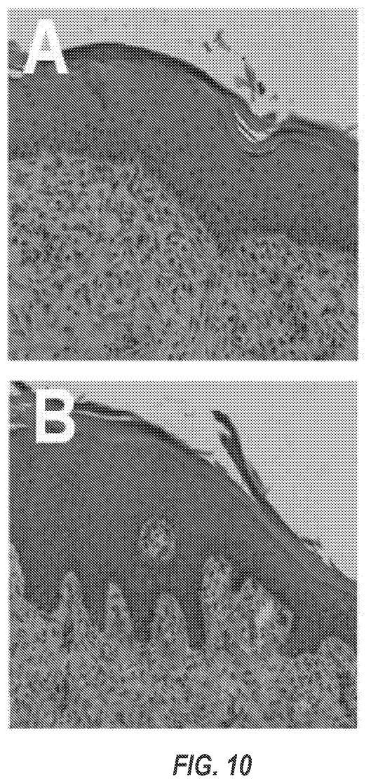

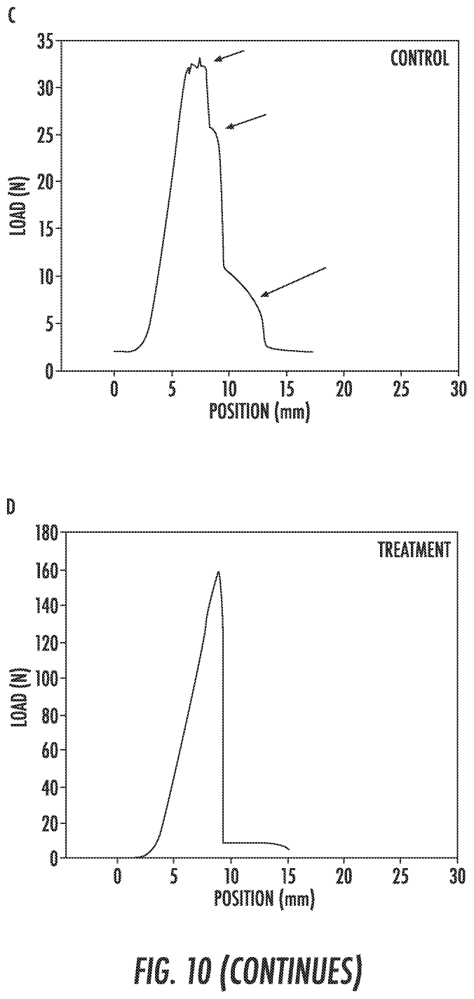

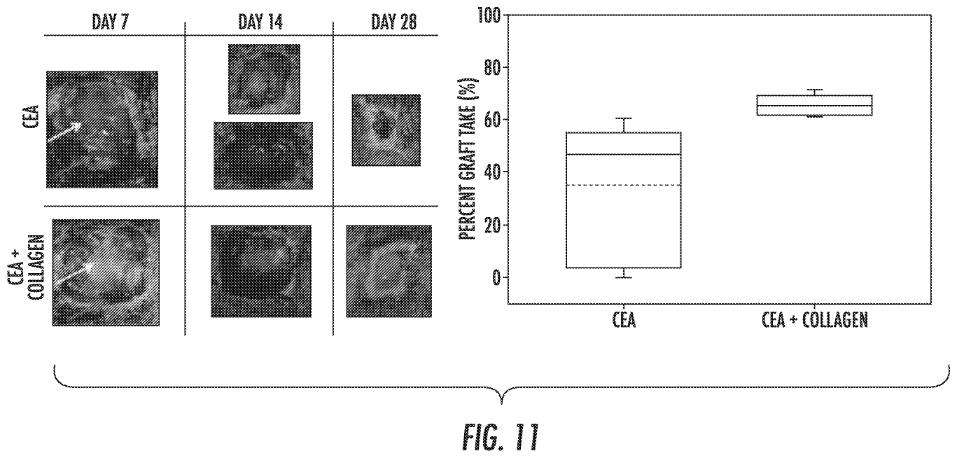

IL-6 and KGF were increased when full-thickness skin substitutes were made with rete ridges compared to flat controls (FIG. 9).

Methods. Rete ridge and flat dermal analogs will be fabricated. Cultured epithelial autografts will be formed by inoculating primary human keratinocytes (multiple strains will be used) at a density of 2×10⁴/cm² onto culture with medium exchanged every other day (Medium 153 (Sigma) supplemented with 0.2 vol. % bovine pituitary extract, 1 μg/mL epidermal growth factor (EGF), 5 mg/mL insulin, 0.5 mg/mL hydrocortisone (HC) and 1% pen-strep-fungizone(PSF)). Cell sheets will be placed onto flat and rete ridge dermal substitutes and cultured at the air-liquid interface for a total of 14 days in custom formulated skin medium (Blackstone et al., Tissue Eng Part A. 2014; 20(19-20):2746-2755; Blackstone et al., Adv Wound Care (New Rochelle). 2012; 1(2):69-74). Medium will be collected at days 1, 4, 7, and 14 for ELISA (KGF, IL-6, IL-1a, IL-1b, and periostin). Biopsies will be collected at days 1, 4, 7, and 14 and processed for immunostaining to localize Ki67, involucrin, loricrin, p63, β1 integrin, keratin 5, and keratin 14. Biopsies will also be collected for quantitative analysis of gene and protein expression (involucrin, loricrin p63, β1 integrin, keratin 5, keratin 14, delta 1 and connexin 43). Surface electrical capacitance and transepidermal water loss will also be quantified at days 4, 7 and 14 to evaluate barrier function (Powell et al., Biomaterials. 2008, 29(7);834-43; Powell et al., J Biomed Mater Res A. 2008; 84(4):1078-86; Powell et al., Biomaterials. 2006,27(34); 5821-7).

### Example 4. Basement Membrane Production, Maturation and Strength as a Function of Rete Ridge Architecture

CEA will be cultured with flat and rete ridge-containing dermal substitutes at the air-liquid interface. At multiple time points within the culture, basement membrane protein localization and thickness/total quantity will be quantified using immunohistochemistry and western blotting. Hemidesmosotne presence and maturation will be examined in tissue biopsies via transmission electron microscopy analysis. Larger CEA-dermal substitute biopsies will be collected and mechanically tested. Force required to cause tissue delamination and blistering will be quantified in each of these tests.

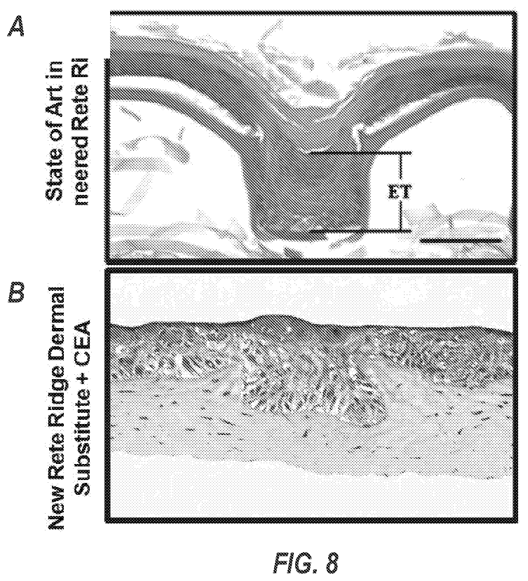

CEA can be applied to the disclosed dermal substitutes in vitro and conform to the rete ridges (FIG. 8).

Methods. CEA-dermal substitute composites will be made as above however the total size of the construct will be increased to 5×5 cm. Constructs will be cultured at the air-liquid interface for up to 14 days with tissue biopsies collected at days 4, 7 and 14. Constructs will analyzed via histology and western blotting for integrin α6 β4, integrin β1, plectin, collagen IV, and laminins 5 and 6. Hemidesmosomes and anchoring filaments will be examined via TEM following methodologies previously described (Dos Santos et al., Matrix Biol. 2015; pii:S0945-053X(15)00060-8). At day 4. 7 and 14, biopsies (40 mm×5 mm) will also be collected for mechanical analyses. A subset of the biopsies will be tensile tested and del amination events per test recorded (delamination events result in a shoulder on the force-position curve). In addition, shear stress and peel off tests will be performed to directly quantify strength of the dermal-epidermal junction. Shear stress will be performed using a dynamic mechanical analyzer with a parallel plate (8 mm in diameter) set up. Peel off tests will be performed by affixing a polymer block to the surface of the epidermis and applying a normal force until the epidermis is removed from the dermis which is affixed to a stationary plate below. Any tests where either adhesive fails will be discarded. Maximum load before failure will be recorded and is a direct representation of the dermal-epidermal junction strength.

### Example 5. Role of Rete Ridge Architecture on Initial Engraftment and Basement Membrane Formation/Maturation in an Autologous Porcine Burn-CEA Model

Thin split-thickness autografts will be collected from each pig in the study to develop CEA and general a pool of allogenic fibroblasts from which to engineer the rete ridge dermal substitutes. Full-thickness burns will be created, eschar excised, and wounds treated with CEA+flat or rete ridge dermal substitutes. Grafts will be dressed with a dressing, which will later be removed and the graft take evaluated using photography and digital image analysis. Biopsies will be collected and evaluated for basement membrane production and maturation. in addition, non-destructive mechanical analysis will be performed to quantify the strength of the interface between the dermis and epidermis.

Porcine CEA was successfully engrafted to excised burn wounds with significant increase in engraftment rates when CEA was combined with a rough collagen dermal template (FIG. 11).

Methods. Small split-thickness autografts (2 in×2 in) will be collected from each pig and fibroblasts/keratinocytes isolated following procedures previously described. Isolated keratinocytes will be cultured on collagen coated culture vessels with supplemented MCDB153 (Sigma Aldrich). Fibroblasts will be cultured in OptiMFM supplemented with 10% FBS and 1% PSF. Red. Duroc pigs will be shaved and scrubbed, followed by 8-1×1 inch full-thickness thermal injuries created on the dorsum following protocol previously described (Kim et al., Plast Reconstr Surg. (in Press 2015)). Burn eschar will be excised and treated with CEA flat or rete ridge dermal substitutes (CEA alone and split-thickness skin grafts will serve as a control). Rete ridge structures which promoted the greatest epidermal viability and basement membrane formation will be utilized. Grafts will be dressed and, after 7 days, dressings will be removed and graft take evaluated using photography and digital image analysis. Biopsies will be collected at days 7, 14, 21, 30, 45 and 60 and evaluated for basement membrane production (immunohistochemistry and western blotting for collagen IV, laminin 5, laminin-3A32, and integrins α3β1 and α6β4) and maturation (via TEM). In addition, non-destructive mechanical analysis via cutometry (BTC 2000) will be performed following a protocol (Bailey et al., Dermatol Surg. 2012; 38(9):1490-6) to quantify the strength of the interface between the dermis and epidermis.

### Example 6. Autologous Porcine CEA contraction and Biomechanics as a Function of Rete Ridge Structure

CEA+rete ridge dermal substitutes and CEA alone will be grafted to full-thickness burns on Red Duroc pigs following eschar removal. Pigs will be evaluated post-operation for graft take, scar/skin contraction, volume, biomechanics and anatomy.

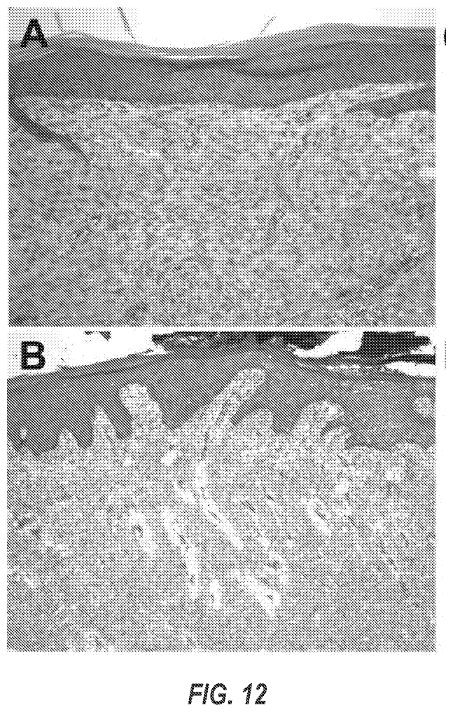

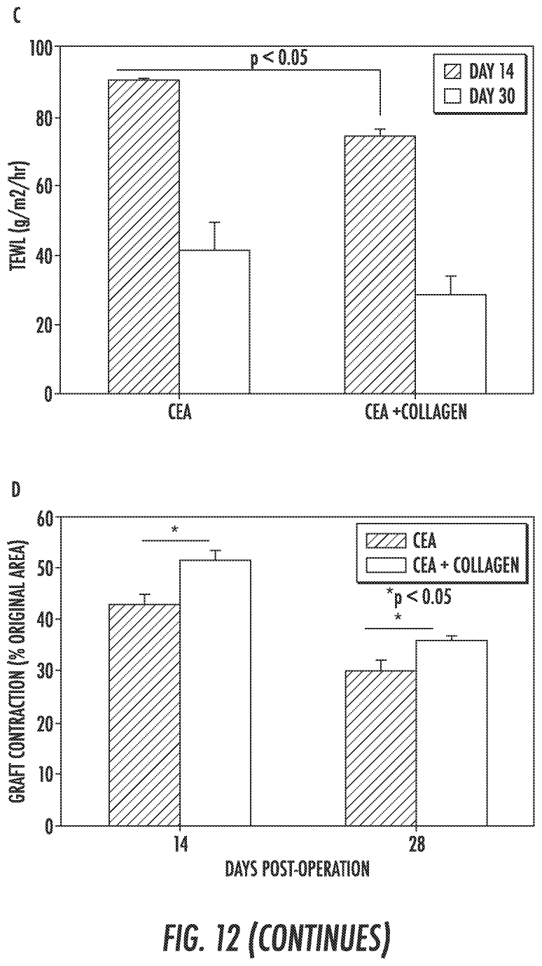

Decreased contraction, more rapid epidermal barrier formation and improved rete ridge formation were observed using a porcine CEA+collagen model (FIG. 12).

Methods. CEAs, rete ridge dermal substitutes, and full-thickness bum injuries will be formed (8 wounds/grafts per pig). CEAs alone and flat dermal substitutes will serve as controls (all within the same pig) Initial engraftment rates will be quantified using digital image analysis at day 7 when dressings are removed. At days 15, 30, 60 and 90, grafts will be qualitatively assessed using the Vancouver scar scale (Bailey et al., Dermatol Surg. 2012; 38(9):1490-6). Quantitative, non-destructive measures of scar size, scar mechanics (elasticity, hardness, and stiffness), TEWL/barrier function (g of water lost per m2 per hour) and scar perfusion will be performed using digital photography, torsional ballistometry (Diastron Ltd, Broomall, Pa.), cutometry (DermaLab Combo, cyberDerm Inc., Media, Pa.), evaporimetry (TEWL; DermaLab), and Laser Flowmetry (PeriMed Instruments). In addition to the non-destructive assays, punch biopsies (6 mm) will be collected from select grafts and evaluated using histology, immunostaining and qPCR/westem blotting (picrosirius red staining, collagen type I, collagen type III, von Willebrand Factor (vWF), CD31, Ki67, involucrin, loricrin, a-smooth muscle actin, macrophage-1 antigen)

### Example 7

### Materials and Methods

Scaffold Preparation. Scaffolds were electrospun with acid-soluble collagen from comminuted bovine hide (Kensey Nash, Exton, Pa.), Collagen was solubilized in hexafluoropropanol (HFP; Oakwood Chemical, Estill, S.C.) with a concentration of 10% wt./vol. Solubilized collagen was ejected via syringe pump at a rate of 4.3 mL/hr, with a potential of 30 kV, onto a grounding plate positioned 18 cm from the tip of the needle. Scaffolds were physically crosslinked using dehydrothermal treatment at 140° C. for 24 hr and stored in a vacuum until use. Scaffolds were prepared for cell culture as previously described (Drexler, et al., Acta Biomater. 7, 1133-1139 (2011)), with chemical crosslinking in a solution of 5 mM N-(3-dimethylaminopropyl)-N′-ethylcarbodiimide hydrochloride (EDC; Sigma, St. Louis, Mo.) in absolute ethanol. The scaffolds were then disinfected with 70% ethanol, rinsed with phosphate buffered saline (PBS), HEPES buffered saline (FIBS) and cell culture medium.

Cell Culture. Human dermal fibroblasts (HF) and epidermal keratinocytes (HK) were isolated from surgical discard tissue. HF and HK at passage 1 were used for all experiments. IfF were cultured with DME (Invitrogen, Grand Island, N.Y.) supplemented with 4% fetal bovine serum (FBS; Invitrogen), 5 μg/mL bovine insulin (Sigma), 0.1 mM L-ascorbic acid-2-phosphate (Sigma), 0.5 μg/ml hydrocortisone (Sigma) and 10 ng/ml epidermal growth factor (EGF; PeproTech, Rocky Hill, N.J.). HK were cultured with 153 (Sigma) supplemented with 0.2 vol % bovine pituitary extract (Gemini BioProducts, West Sacremento, Calif.), 5 μg/mL bovine insulin, 0.5 μg/ml hydrocortisone, and 1 ng/ml EGF.

Formation of Engineered Skin. Crosslinked and rinsed collagen scaffolds were inoculated with HE at 5×10⁵ cells/cm² and incubated for 5 days at 37° C. and 5% CO₂. The constructs were cultured in DME (Sigma) supplemented with 5 μg/ml bovine insulin, 0.1 mM ascorbic acid-3-phosphate, 0.5 μg/ml hydrocortisone, 10 ng/ml EGF, 20 pM triiodothyronine (Sigma), 0.76 nM progesterone (Sigma), 2 μg/ml linoleic acid (Sigma), and 1 mM strontium chloride (Sigma). After 5 days of daily medium changes, MK were inoculated onto these dermal constructs. To produce ridged samples, immediately prior to HK inoculation, dermal constructs were treated with an Ultrapulse® fractional carbon dioxide laser (FXCO₂, Lumenis Inc., San Jose, Calif.), A DeepFX™ handset was used to deliver 5 mJ at a density of 25%. Both untreated (flat) and laser treated (ridged) samples were inoculated with HK at 1×10⁶ cells/cm² and incubated, submerged, overnight. The rapid pulses of energy heat water in the tissue causing ablation and ridge formation. The properties of the ridge can be tuned by altering the amount of energy. The following day, in vitro culture day 1, engineered skin was then lifted to the air-liquid interface, on permeable cotton pads (Whatman, GE Healthcare, Milwaukee, Wis.) supported by perforated stainless steel frames. From the third day after HK inoculation until the conclusion of in vitro culture, EGF and progesterone were excluded from the culture medium to facilitate epidermal differentiation.

Viability and morphology were assessed pre- and post-lasering, and at days 1, 5 and 11 after HK inoculation. Metabolic activity was assessed from 4 mm biopsy punches collected from each graft (n=6 per condition) via MIT assay (Sigma). Additional biopsies were embedded in OCT compound for cryosectioning and staining. Post-lasering, biopsies were fixed in 4% PEA (Sigma) and either processed for scanning electron microscopy (SEM, FEI Nova 400 NanoSEM) or for whole mount immunostaining. Surface hydration (SEC) was also assessed at in vitro culture days 5 and 11 with a NOVA dermal phase meter (n=6 per condition; DPM 9003; NOVA Technology, Portsmouth, N.H.). Lower SEC values indicate improved epidermal differentiation and barrier function. MTT (absorbance) and SEC (DPM units) were reported as mean±standard error of the mean.

Engineered Skin (ES) Grafting to Immunocompromised Mice. 2 cm×2 cm excisional wounds were created on the dorsum of immunodeficient mice (n=15 per condition; Foxn1^{mu/mu}, Jackson Labs, Bar Harbor, Me.) and skin was removed from the panniculus. ES at in vitro culture day 10 or 11 were cut into 2 cm×2 cm pieces and grafted to the wound site. Grafts were dressed with N-terface® (Winfield Laboratories, Inc., Richardson, Tex.) and antibiotic ointment-coated gauze and sutured into place with a stent tie-over dressing (Swope, et al., Wound Repair Regen. 10, 378-386 (2002); Harriger, et al., J Biomed Mater Res 35, 137-145 (1997)). The site was covered with Tegaderm™ (3M™, St. Paul, Minn.) and Coban™ (3M™). Animals and dressings were assessed daily. All dressings and sutures were removed two weeks post-grafting,

Animal Data Collection and Analysis. Grafts were evaluated at the time of grafting on in vitro culture day 11 (week 0) and at weeks 2 and 4 post-grafting. Grafts were photographed at weeks 2 and 4 and normalized for brightness. Graft area was traced manually at weeks 0, 2 and 4, quantified using imageJ, and reported as percent of original wound area standard error of the mean. Graft healing was assessed at weeks 2 and 4 via transepidermal water loss (TEWL) using a Tewameter® TM 300 probe (Courage+Khazaka Electronic GmbH, Köln, Germany), At week 2, measurements were taken at least 3 hours after bandage removal. TEWL was also measured on the dorsum opposite of the grafts of 12 mice to establish a baseline. Additionally at weeks 2 and 4, six to seven animals per graft condition were euthanized for tissue collection. Biopsies from each graft were frozen in OCT compound for cryosectioning. A dogbone-shaped punch was used to remove a biopsy from each graft for tensile testing (described below).

Immunostaining. To evaluate the effect of laser treatment on the morphology and cell population of the dermal component, fixed day 0 flat and post-laser ridged samples were immunostained using DAPI (ThermoFisher Scientific, Waltham, Mass.) and AlexaFluor® 488 phalloidin (Invitrogen). Frozen OCT-embedded samples were sectioned at a thickness of 7 μm. For ridged samples, 60 serial sections and 90 serial sections were acquired for in vitro and in vivo samples, respectively. Slides were acetone-fixed and assessed via light microscopy, and representative sections were utilized for immunostaining. At in vitro culture days 5 and 11, samples were double immunostained using monoclonal antibodies mouse anti-pan-cytokeratin (Santa Cruz Biotechnology, Dallas, Te) and mouse anti-laminin-5 (Abeam, Cambridge, Mass.). ES at in vitro day 5 (week −1), prior to grafting at week 0, and grafts at weeks 2 and 4 post-grafting were immunostained with monoclonal antibodies mouse anti-laminin-5, rabbit anti-Ki67 (Abeam) and goat anti-loricrin (Santa Cruz). Primary antibodies were detected with AlexaFluor® secondary antibodies (Invitrogen), Fluorescence was imaged using an Olympus FV1000 Filter confocal microscope. Four grafts were assessed per condition and four non-overlapping regions were captured at 40× for quantification. All measurements were reported as the average per field of view±standard error of the mean. Basement membrane length was quantified via Image) (NIH) as the average distance positive for laminin-5 staining per field of view. Epidermal area was also quantified with ImageJ as the average area above positive laminin-5 staining. To quantify keratinocyte proliferation, Ki67+ cells above the basement membrane (determined via laminin-5) were counted and reported as the average number of Ki67-f+ cells per field of view.

Tensile Testing. Graft mechanical properties were evaluated at weeks 2 and 4 post-grafting. Uniformly shaped samples were obtained with a dog-bone shaped punch with 10 mm gauge length and 3 mm gauge width. Samples were loaded into a tensile tester (TestResources 100R, Shakopee, Minn.) and strained until failure at a rate of 2 mm/sec. Maximum load, linear stiffness and area under the load vs. position curve were calculated and reported as mean±standard error of the mean.

Statistical Analyses. Data were analyzed with Minita.b (Minitab, Inc., State College, Pa.). Difference between conditions was evaluated with One Way analysis of Variance (ANOVA) with a post-hoc test of Tukey. Statistical significance was determined with a p value of 0.05.

### Results

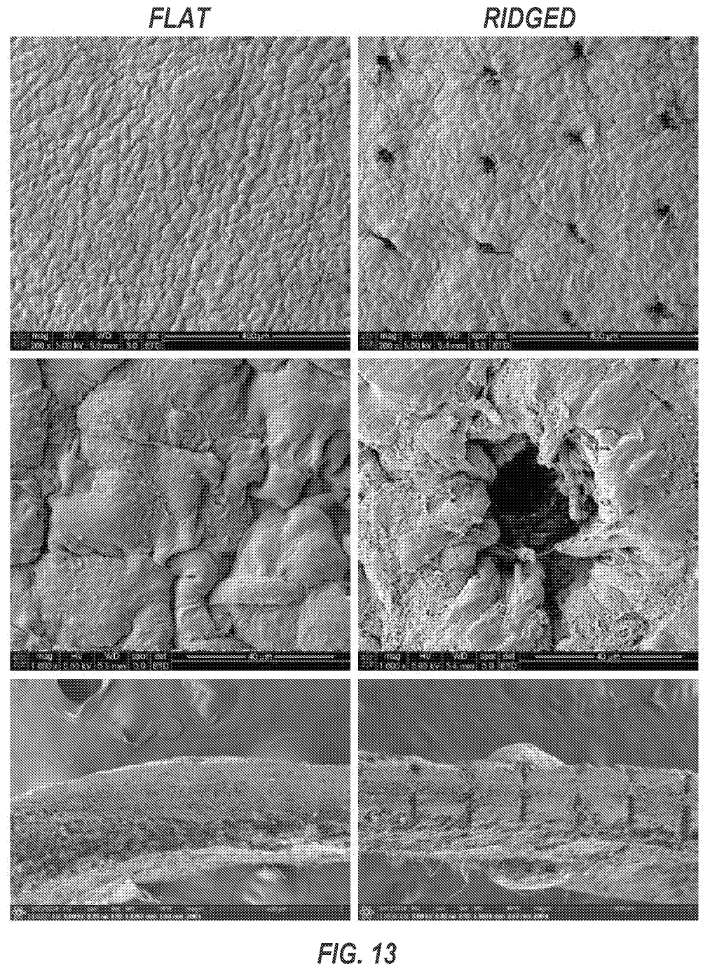

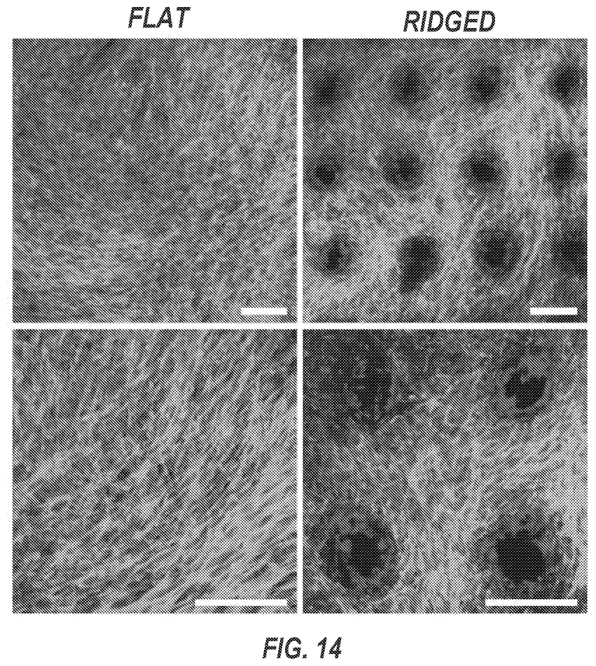

Engineered Skin Analysis, Morphology of the dermal component was assessed following laser treatment and compared to untreated, flat, samples. SEM displayed a uniform layer of tightly packed fibroblasts on flat samples, while ridged samples presented a similar cell layer broken up by laser ablated regions surrounded by areas of damaged cells and collagen fibers (FIG. 13). The ablated regions extended to approximately ⅔ the depth of the dermal construct. Immunostaining for nuclei and F-actin in laser treated samples revealed 50-100 μm diameter holes at the surface, surrounded by 20-50 μm wide zones absent of F-actin (FIG. 14).

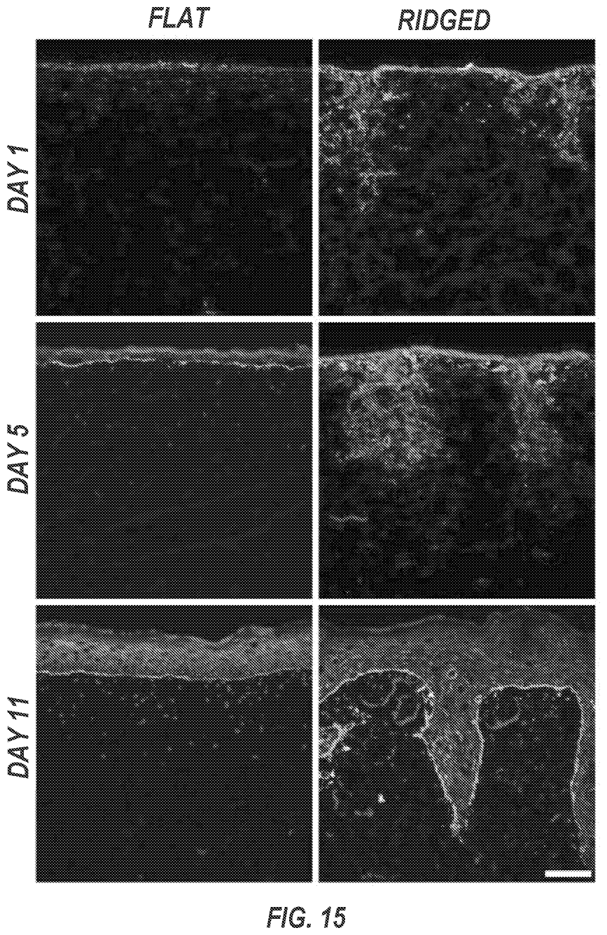

Laminin and cytokeratin staining was performed 1, 5 and 11 days after keratinocyte inoculation (FIG. 15). Little to no positive laminin staining was observed in flat samples on day 1. Laminin significantly increased in presence by day 5 and was a continuous layer between epidermal and dermal cells at day 11. More laminin positive staining was noted at all time points in ridged samples. At days 1 and 5, keratinocytes were more diffuse where the laser had penetrated the dermal construct and only formed a tight, discrete layer in between ablation zones. By day 11, keratinocytes in ridged samples densified to form discrete projections into the dermis, with an intense laminin layer separating the compartments.

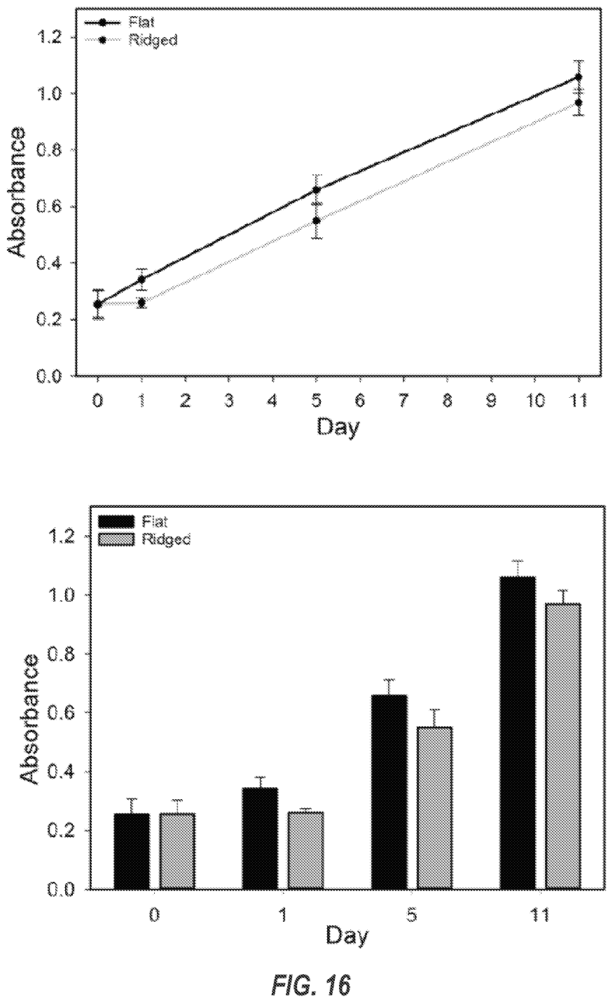

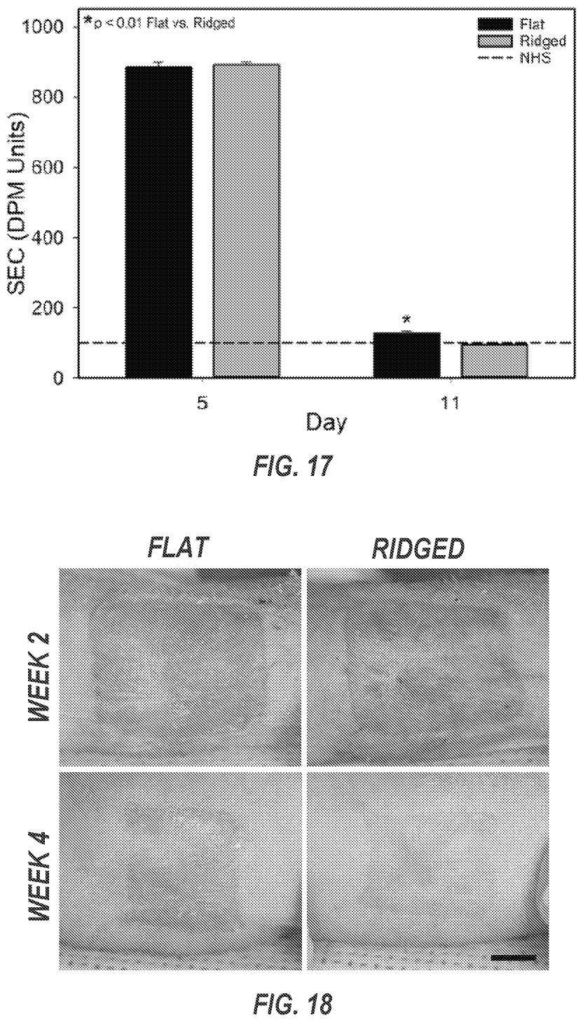

Cell viability was assessed over the culture period and only a small, non-significant decrease was noted in ridged samples versus flat samples after HK inoculation (FIG. 16). Epidermal differentiation quantified via SEC was similar in both conditions and expectedly high at day 5 (FIG. 17). At day 11, SEC for both groups decreased significantly and was similar to that of native human skin, though ridged samples displayed significantly lower SEC values than flat samples.

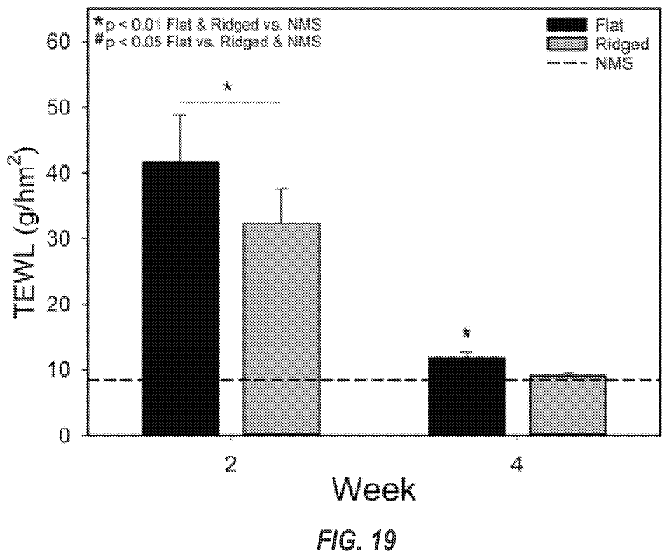

Macroscopic Graft Assessment. Two weeks after grafting ES to immunodeficient mice, dressings were removed and grafts were assessed at weeks 2 and 4. Flat and ridged grafts were similar in appearance and showed a small amount of dehiscence at the graft-mouse skin interface (FIG. 18). ES grafts contracted significantly over time, though at similar rates. Hat and ridged grafts had contracted respectively to 89.5±2.73% and 88.3±2.48% at week 2, and to 39.1±4.78 vs. 36.1±3.66% at week 4 (data not shown). At week 2, transepidermal water loss (TE of both groups was significantly increased from normal mouse skin, and ridged grafts were only slightly decreased from flat grafts (FIG. 19). TEWL for both groups decreased over time and at week 4 ridged grafts were statistically similar to normal mouse skin, while flat grafts had significantly higher TEL than ridged grafts and normal mouse skin.

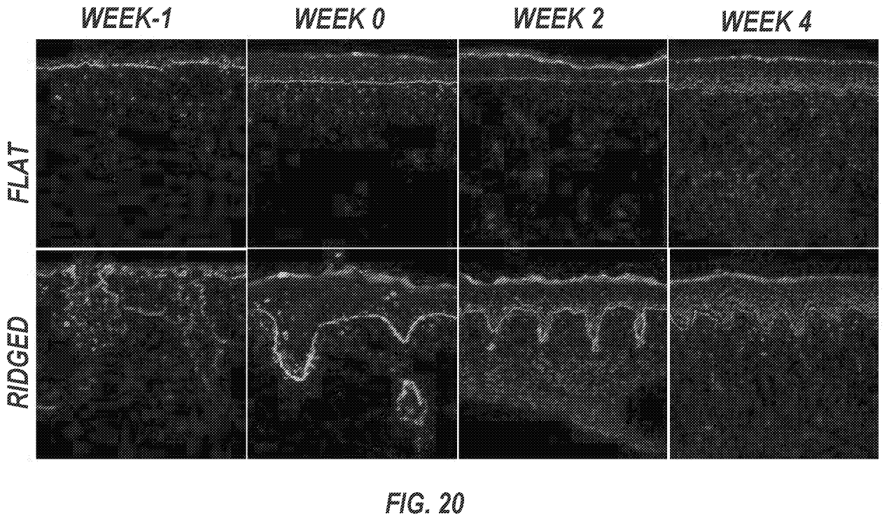

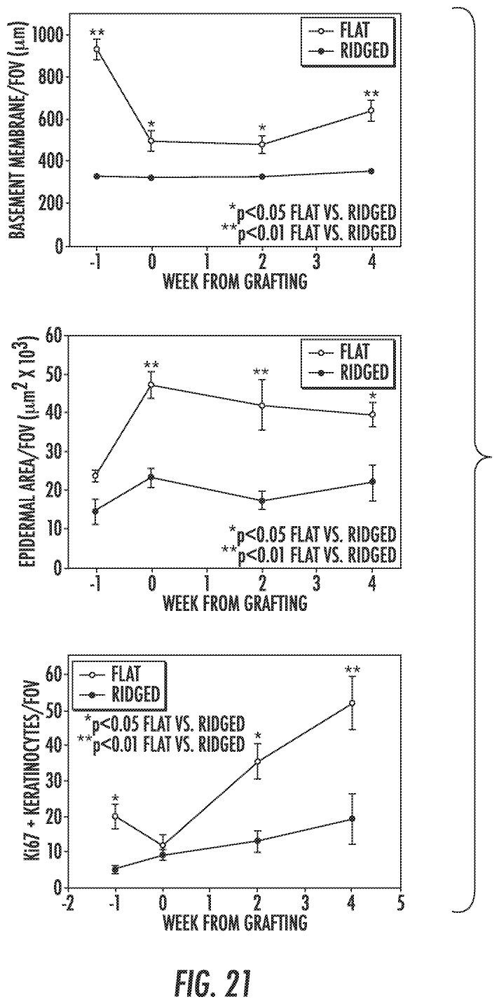

Engineered Skin Graft Composition. Grafts were immunostained for laminin-5 and Ki67 1 week prior to grafting (week −1), at the time of grafting (week 0) and at weeks 2 and 4 post-grafting (FIG. 20). Five days after lasering and inoculating keratinocytes, both basement membrane length and number of KI67+keratinocytes were significantly increased in ridged samples (FIG. 21). At the time of grafting, basement membrane length had decreased, though, was still significantly greater than flat samples (FIG. 21A). By the time of grafting, epidermal area of ridged samples was twice that of flat samples and remained significantly greater than flat samples for the duration of the experiment (FIG. 21B). The number of proliferative keratinocytes decreased from week −1 in ridged samples at the time of grafting, though significantly increased post-grafting in comparison to flat grafts (FIG. 21C).

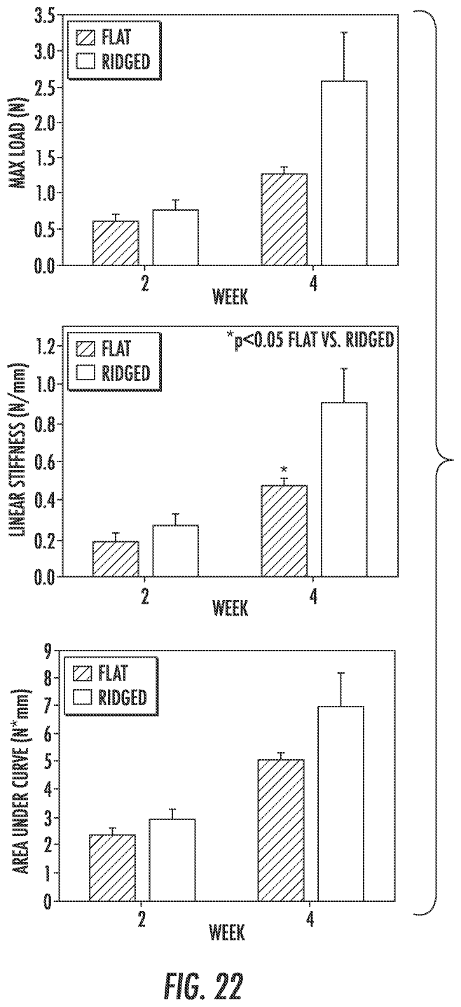

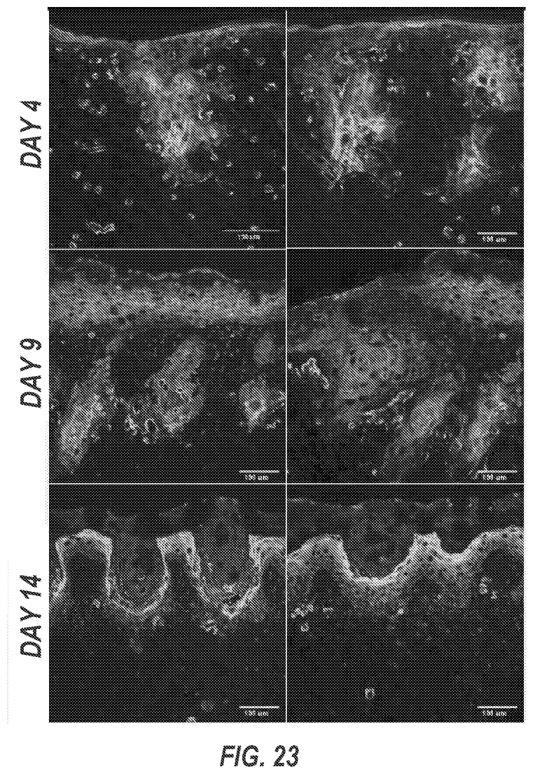

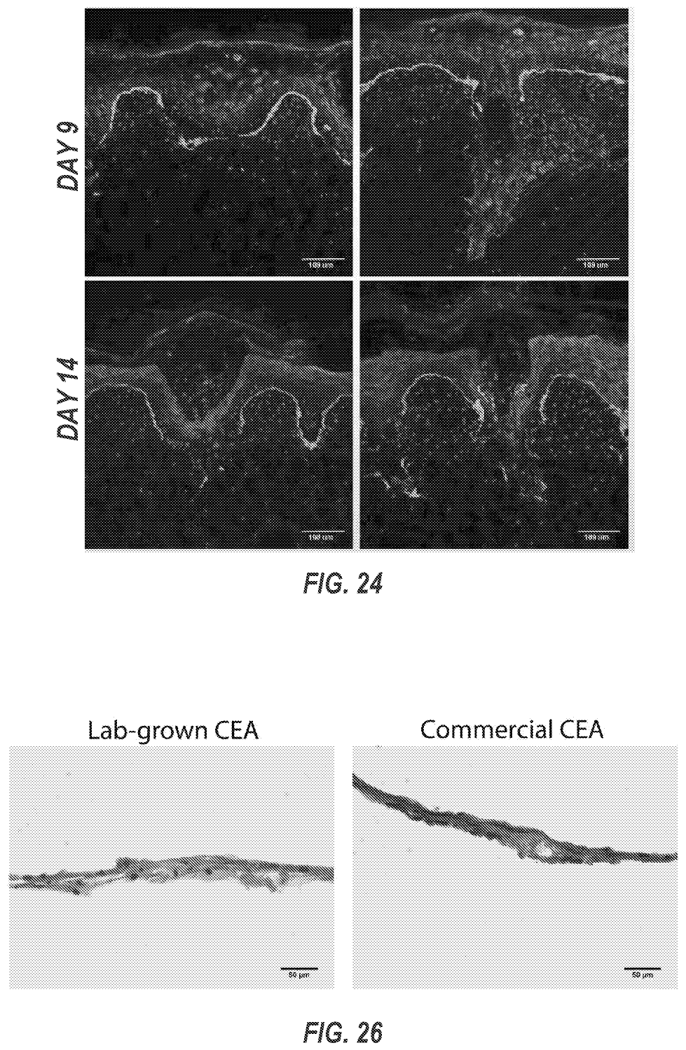

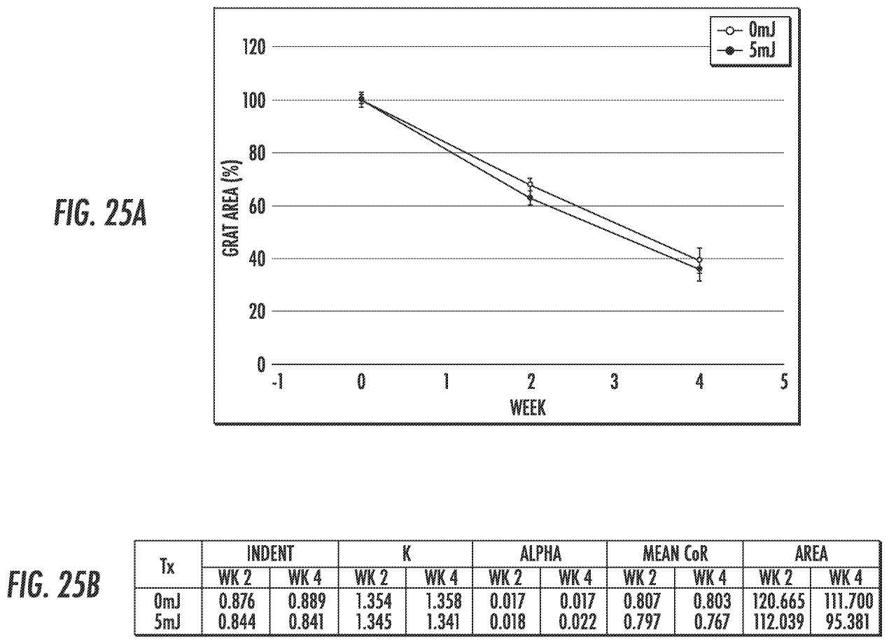

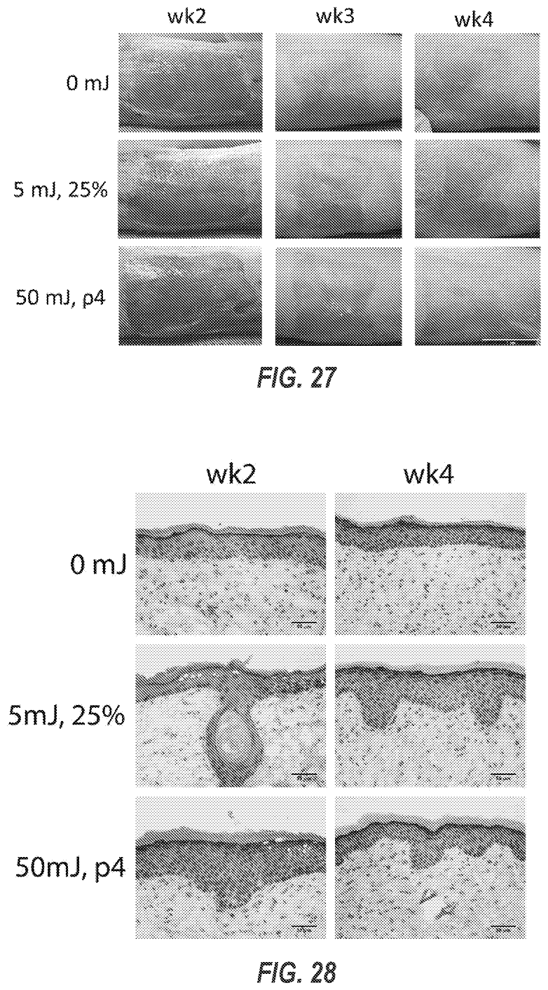

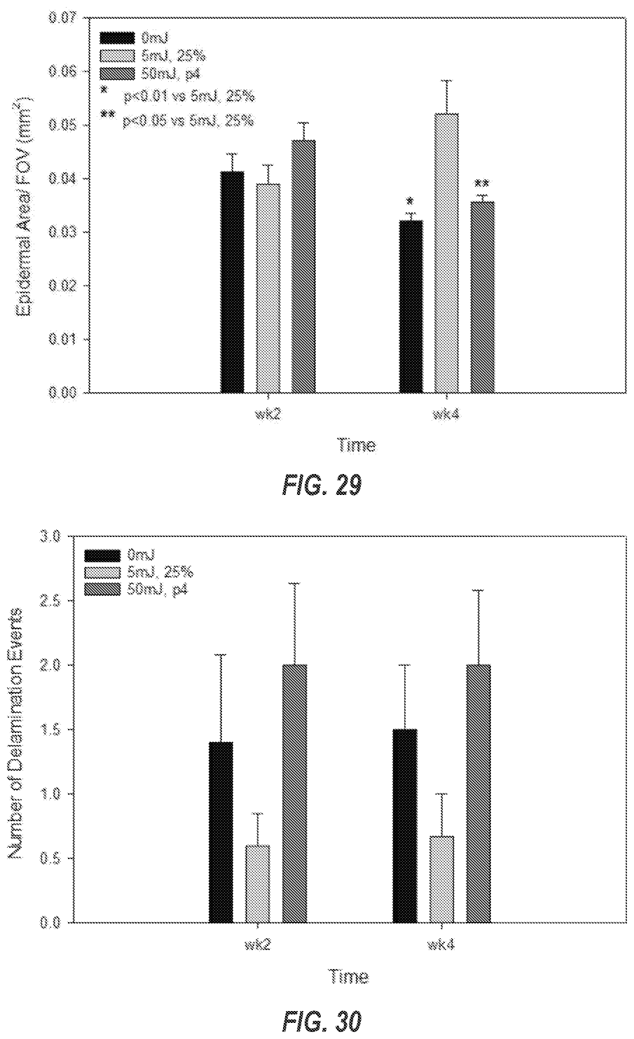

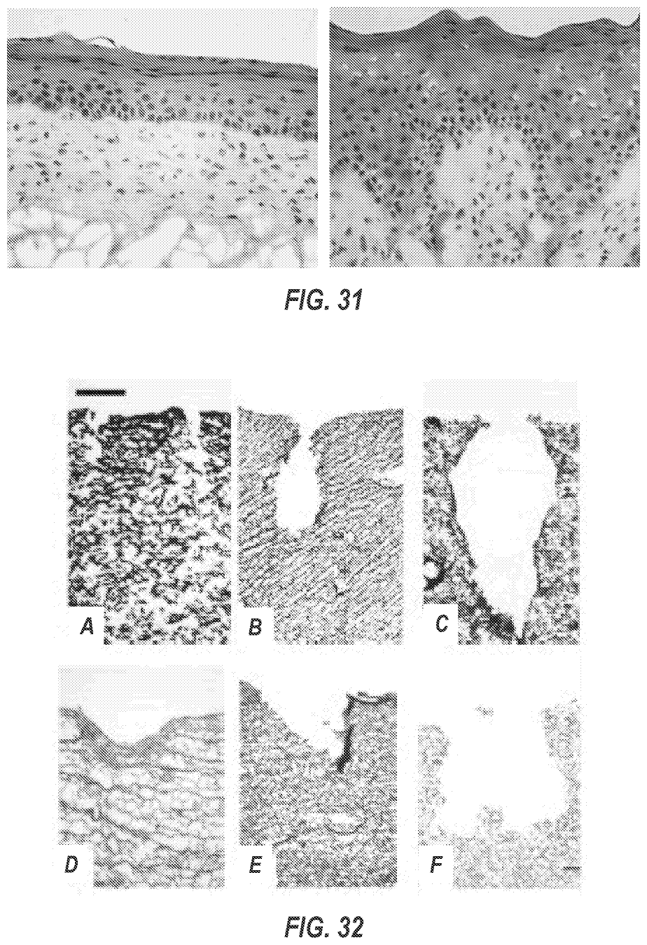

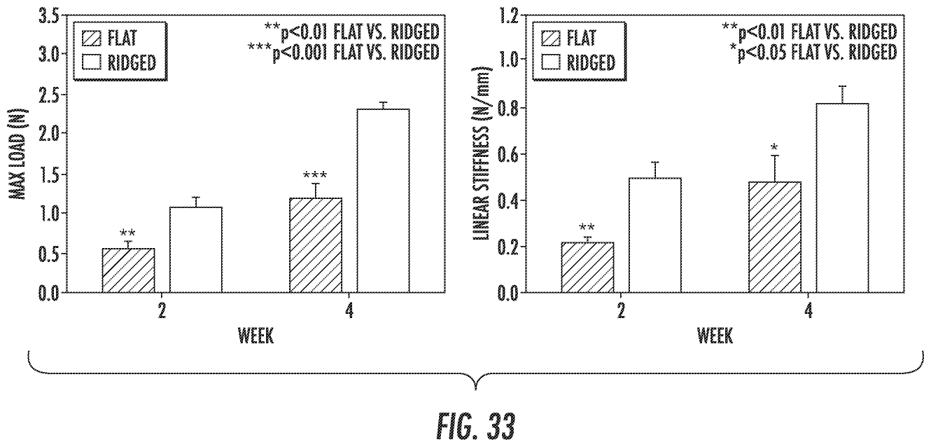

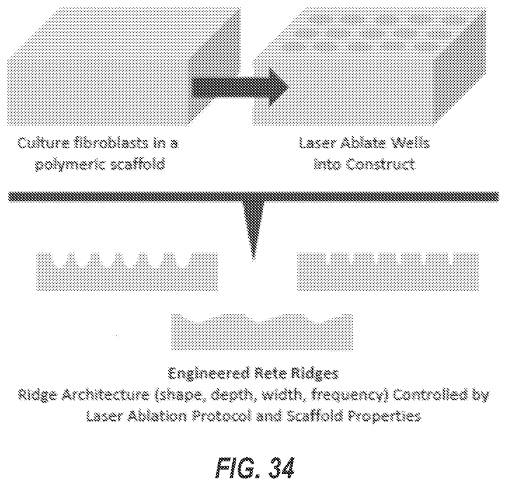

In Vivo Engineered Skin Mechanics. Testing dogbone-shaped samples of grafts at weeks 2 and 4 to failure showed increases in maximum load and area under the curve for ridged samples at week 4, though these increases were non-significant (FIG. 22). Linear stiffness of ridged grafts was found to be significantly increased at week 4, with a 1.96-fold increase over flat grafts.

### Example 8. Combinatorial Use of CEAs with Dermal Substitutes Containing Dermal Papilla-Like Structures

Methods: Human CEAs were fabricated by culturing primary epidermal keratinocytes for 19 days followed by release from plastic by brief exposure to dispase. Dermal substitutes were created by seeding electrospun collagen scaffolds with primary human fibroblasts at 500,000/cm². Dermal substitutes were cultured for 5 days prior to creation of papillae-like structures. The surface of the engineered dermis was CO₂ laser ablated to form a series of protrusions on the surface to mimic dermal papillae structure. On the day of surgery, CEAs were placed on the dermal substitutes containing papillae-like structures (PS-PLS) and immediately grafted to full-thickness surgical wounds in athymic mice; flat substitutes served as controls. Graft area, mechanical properties and structure were assessed 2 and 4 weeks post grafting.

Results: The DS-PLS promoted the interdigitation of the dermis and epidermis. Though the presence of the papillae-like structures did not significantly alter graft contraction, the epidermis was significantly thicker in this group versus flat controls. In addition, basement membrane deposition was increased in the DS-PLS versus controls and resulted in less delamination of the epidermis and dermis upon tensile testing.

Conclusions: Dermal papillae play important roles in skin function; in particular, they enhance epidermal-dermal adhesive strength . The results of this study suggest that engineering dermal papillae-like structures into dermal substitutes can promote interdigitation of CEAs and the dermis and lead to improved epidermal-dermal adhesion.

### Example 9. Engineered Rete Ridges Enhance Epidermal Thickness and Establishment of Barrier Function in Skin Substitutes

Methods: Human primary fibroblasts (HT) and keratinocytes (HK) were isolated from skin with IRB approval. ES was fabricated by seeding HF onto an electrospun collagen scaffold at 500,000 cells/cm². After 5 days in culture , wells were created at the surface by laser ablation (˜175 μm wide, 250 μm deep, spaced 175 μm apart). Constructs without wells served as controls. HK were seeded at 1,000,000 cells/cm² and the ES was cultured at the air-liquid interface for 10 days. At days 1, 5 and 10 in vitro, surface electrical capacitance (SEC) was measured and biopsies were collected from for histological analysis and Ki67 immunostaining. At day 10, engineered skin was grafted to full-thickness wounds in athymic mice. At weeks 2, 3 and 4, barrier function was assessed using a transepidermal water loss meter and graft area was quantified via planimetry. Six mice were euthanized at each time point and tissue collected for histological evaluation and gene expression analysis. Analysis of delta-like 1, leucine-rich repeat containing G protein-coupled receptor 6, leucine-rich repeat containing G protein-coupled receptor 1 and axin 2 gene expression will be assessed via real-time qRT-PCR. Histological sections will be stained with keratin 2A, 6, 15 and 16, along with periostin to assess epidermal differentiation and stem-like populations.

Results: The presence of the rete ridges in vitro significant increased epidermal proliferation and basement membrane area. SEC, which is inversely correlated to barrier function, was significantly lower in rete ridge ES at day 10 versus flat ES. Both groups engrafted in vivo with no graft loss. At weeks 2 and 4 in vivo, ridges remained stable and resulted in a thicker epidermis and increased Ki67+proliferative cells versus flat ES.

### Example 10. Engineered Rete Ridges Enhance Basement Membrane Formation and Strength in Skin Substitutes

Methods: Human primary fibroblasts (HF) and keratinocytes (HK) were isolated from skin with IRB approval. ES was fabricated by seeding IIF onto an electrospun collagen scaffold at 500,000 cells/cm². After 5 days in culture , wells were created at the surface by laser ablation (˜175 μm wide, 250 μm deep, spaced 175 μm apart). Constructs without wells served as controls. HK were seeded at 1,000,000 cells/cm² and the ES was cultured at the air-liquid interface for 10 days. At days 1, 5 and 10 in vitro, surface electrical capacitance (SEC) was measured and biopsies were collected from for histological analysis and Ki67 immunostaining. At day 10, engineered skin was grafted to full-thickness wounds in athymic mice and grafts stented with a silicone membrane that was 0.5 mm thick, 2 mm wide and framing a 2×2. cm area. At weeks 2, 3 and 4, barrier function was assessed using a transepidermal water loss meter and graft area was quantified via planimetry. Six mice were euthanized at weeks 2 and 4 and tissue collected for histological evaluation, gene expression analysis and assessment of mechanical properties. Analysis of collagen I, collagen :III and collagen IV gene expression will be assessed via real-time qRT-PCR. Histological sections will be stained with collagen IV, laminin-5, collagen VII, integrin β1 and integrin a to assess basement membrane formation and epidermal attachment to the basement membrane. Dogbone shaped specimens at weeks 2 and 4 will be tensile tested to failure at 2 mm/sec to quantify ultimate tensile strength and linear stiffness. in addition, a peel-off test will be utilized to assess the strength of epidermal-dermal adhesion.

Results: The presence of the rete ridges in vitro significantly improved skin substitute strength and stiffness post-grafting. Ridges also enhanced the formation of basement membrane in vitro and in vivo.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A dermal substitute comprising:
a substantially planar sheet comprising fibroblasts dispersed within a biocompatible polymer matrix, the substantially planar sheet having a plurality of protrusions extending from at least one surface of the substantially planar sheet;
wherein the plurality of protrusions are sized to improve a dermal graft outcome.

2. The dermal substitute of claim 1, wherein the biocompatible polymeric matrix comprises collagen.

3. The dermal substitute of claim 1, wherein each of the plurality of protrusions have a length of at least 50 μm, such as a length of from 50 μm to 750 μm.

4. The dermal substitute of claim 1, wherein each of the plurality of protrusions have a width of at least 50 μm, such as a width of from 50 μm to 750 μm.

5. The dermal substitute of claim 1, wherein each of the plurality of protrusions have a height of at least 10 μm, such as a height of from 10 μm to 750 μm.

6. The dermal substitute of claim 1, wherein the plurality of protrusions spaced apart by an average spacing along an axis of least 50 μm, such as a width of from 50 μm to 750 μm.

7. The dermal substitute of claim 1, wherein the dermal graft outcome is selected from the group consisting of:
increasing a rate of epidermal barrier formation,
increasing an amount of rete ridge formation,
increasing an amount of epidermal-dermal tissue chemical communication,
increasing an amount of basement membrane protein deposition,
increasing an amount of interfacial strength,
decreasing an amount of transepidermal water loss (TEWL),
decreasing an amount of graft contraction; and
combinations thereof
as compared to a cultured epithelial cell graft or a dermal substitute lacking the plurality of protrusions.

8. The dermal substitute of claim 1, wherein the substantially planar sheet comprises an inner surface and an outer surface, and wherein the plurality of protrusions extend from the inner surface of the substantially planar sheet.

9. The dermal substitute of claim 8, further comprising a population of epithelial cells disposed on the outer surface of the substantially planar sheet.

10. A method of treating a skin wound on a subject, the method comprising contacting a skin wound with the dermal substitute of claim 1.

11. The method of claim 10, wherein the skin wound comprises a burn.

12. A method of treating a skin wound on a subject, the method comprising:
contacting a skin wound with a substantially planar sheet comprising fibroblasts dispersed within a biocompatible polymer matrix,
wherein the substantially planar sheet has an inner surface and an outer surface,
wherein the plurality of protrusions extend from the inner surface of the substantially planar sheet, and
wherein the inner surface of the substantially planar sheet is positioned in contact with the skin wound.

13. The method of claim 12, wherein the substantially planar sheet further comprises population of epithelial cells disposed on the outer surface of the substantially planar sheet.

14. The method of claim 13, wherein the epithelial cells are disposed on the outer surface of the substantially planar sheet prior to contacting the portion of skin with the dermal substitute.

15. The method of claim 13, wherein the population of epithelial cells comprises a skin graft, such as an autologous skin graft, an isogenic skin graft, an allogenic skin graft, or a xenogenic skin graft.

16. The method of claim 10, wherein the method increases an amount of a skin growth or repair factor comprising any one or more of keratinocyte growth factor (KGF), IL-6, IL-1a, IL-1b, periostin, Ki67, involucrin, loricrin, p63, β1 integrin, keratin 5, keratin 14, delta 1 or connexin 43.

17. A method of preparing a dermal graft for transplantation, the method comprising culturing fibroblasts dispersed within a biocompatible polymer matrix,
wherein the biocompatible polymer matrix is formed as a substantially planar sheet having an inner surface and an outer surface; and
wherein the inner surface of the substantially planar sheet comprises a plurality of protrusions extending from the inner surface of the substantially planar sheet.

18. The method of claim 17, further comprising crosslinking the biocompatible polymer matrix.

19. The method of claim 17, further comprising exposing the biocompatible polymer matrix to a laser delivering at least 5 mJ.
